(19) United States
(12) Patent Application Publication
(10) Pub. No.: US 2016/0255897 A1
(43) Pub. Date: Sep. 8, 2016

(54) DEVICE FOR ABSORBING AND RETAINING SWEAT

(71) Applicant: No Sweat, LLC, Tonka Bay, MN (US)

(72) Inventors: Justin Johnson, Tonka Bay, MN (US); J.T. Johnson, Tonka Bay, MN (US); Chad Sulheim, Tonka Bay, MN (US)

(21) Appl. No.: 15/157,385

(22) Filed: May 17, 2016

Related U.S. Application Data
(63) Continuation-in-part of application No. 14/535,247, filed on Nov. 6, 2014.
Application No. 14/535,247 is a continuation-in-part of application No. 13/709,913, filed on Dec. 10, 2012, now Pat. No. 8,904,567.
Application No. 13/709,913 is a continuation of application No. 12/416,529, filed on Apr. 1, 2009, now abandoned.
(60) Provisional application No. 61/041,388, filed on Apr. 1, 2008.

(51) Int. Cl. A42B 3/10; A42B 3/04; A61F 13/60; A61F 13/62; A61F 13/82; A61F 13/538

(52) U.S. Cl. CPC A42B 3/10; A61F 13/82; A61F 13/538; A61F 13/60; A61F 13/622; A42B 3/0406; A61F 2013/530481

(57) ABSTRACT

A device for absorbing and retaining sweat that includes at least one moisture-wicking layer and an absorbent core layer. The device may further include a liquid impervious layer, wherein the absorbent core layer is positioned in between the moisture-wicking layer and the liquid impervious layer. The device may be a disposable insert to be positioned on the inside of headwear or it may be a disposable band capable of securing around a user's head.

### CROSS-REFERENCE TO RELATED APPLICATION

This application is a continuation-in-part of U.S. patent application Ser. No. 14/535,247, filed on Nov. 6, 2014, titled DISPOSABLE ABSORBENT INSERT FOR AN ATHLETIC HEAD COVERING, which is a continuation-in-part of U.S. patent application Ser. No. 13/709,913, filed on Dec. 10, 2012, titled DISPOSABLE ABSORBENT INSERT FOR AN ATHLETIC HEAD COVERING, which is a continuation of U.S. patent application Ser. No. 12/416,529, filed on Apr. 1, 2009, titled DISPOSABLE ABSORBENT INSERT FOR AN ATHLETIC HEAD COVERING, which claims the benefit of U.S. Provisional Application No. 61/041,388, filed on Apr. 1, 2008, titled DISPOSABLE ABSORBENT INSERT FOR AN ATHLETIC HEAD COVERING.

### FIELD OF THE DISCLOSURE

The disclosed invention relates to a device for absorbing and retaining sweat. More specifically, it relates to a wearable, disposable device that a user can put into contact with the user's head to prevent sweat from dripping onto the user's face. In one embodiment, the device contains an adhesive layer and is inserted into headwear. In one embodiment, the device is a disposable band that secures around a user's head.

### BACKGROUND OF THE INVENTION

Sports have been a form of hobby, community involvement, and source of exercise for people for decades. The athletic equipment industry is stable and is one that is becoming more and more technologically advanced. Athletes are constantly looking for new ways to enhance their performance and gain a competitive edge over their opponents.

Workers, such as construction workers, make up a large portion of the workforce and work outside during most, if not all, times of the year, regardless of the season. Construction workers frequently have to wear safety helmets or hard hats while they are on site to protect themselves from contact injuries.

A common problem that many athletes and construction workers experience is sweat dripping from their foreheads into their eyes. Inhibiting sweat or any type of moisture from dripping down into the eyes, nose, mouth and other areas of the face will allow the individual to concentrate on the task at hand and not be distracted or uncomfortable during his or her performance. The use of an insert enhances the performance and comfort of an individual during activity by mitigating the possibility of sweat and oil dripping down onto the individual's face during activity and impairing his or her vision.

Currently, there are a variety of moisture absorbing devices for absorbing sweat and other moisture away from the head of an individual during various activities. Such absorbent devices have been used as an insert in a cap, hat, hardhat or sun visor. However, a problem with many inserts is that they are difficult to clean and are not easily replaced when soiled or saturated. Moreover, many disposable inserts are not well shaped for the contours of headwear.

### SUMMARY OF THE INVENTION

In one aspect, a disposable absorbent insert for fitting against a forehead-contacting, curved inside surface of a user's head covering comprises a moisture wicking layer; a liquid absorbent core layer; a liquid impervious layer; an adhesive layer comprising a repositionable pressure-sensitive adhesive; and a release liner layer. A top edge of the insert comprises a plurality of slits to facilitate fitting of the insert onto the curved inside surface of the head covering.

In another aspect, a method of using a disposable absorbent insert on a forehead—contacting, curved inside surface of a user's head covering is described. The insert comprises a liquid absorbent layer, an adhesive layer, and a release liner layer, wherein a top edge of the insert comprises a plurality of slits. The method comprises separating the release liner layer from the adhesive layer and contacting the adhesive layer to the curved inside surface of the head covering while manipulating the top edge of the insert via the slits to fit the insert onto the curved inside surface of the head covering without overlapping areas of the insert.

In one aspect, a disposable, flexible, absorbent band for securing around a user's head comprises at least one moisture wicking layer and a liquid absorbent core layer. The band may have two open ends that tie together or may made at least partially of an elastic material to enable the user to stretch the band around the user's head.

This summary is provided to introduce concepts in simplified form that are further described below in the detailed description. This summary is not intended to identify key features or essential features of the disclosed or claimed subject matter, and is not intended to describe each disclosed embodiment or every implementation of the disclosed or claimed subject matter, and is not intended to be used as an aid in determining the scope of the claimed subject matter. Many other novel advantages, features, and relationships will become apparent as this description proceeds. The figures and the description that follow more particularly exemplify illustrative embodiments.

### BRIEF DESCRIPTION OF THE DRAWINGS

FIG. 1 is a top-down view of one embodiment of the disclosed insert.

FIG. 2 is a top-down view of the disclosed insert of FIG. 1.

FIG. 3 is a top-down view of one embodiment of the disclosed insert illustrating variations in size and shape of the liner.

FIG. 4 is a top-down view of one embodiment of the disclosed insert illustrating variations in size and shape of the liner.

FIG. 5 is a side view of an athletic helmet with the insert of FIG. 1 positioned inside the front portion of the helmet.

FIG. 6 is a side view of a hat with the insert of FIG. 1 positioned inside the front portion of the hat.

FIG. 7 is a side view of a helmet with the insert of FIG. 1 positioned inside the front portion of the helmet.

FIG. 8 is a perspective view of an athletic helmet with the insert of FIG. 1 positioned inside the front portion of the helmet.

FIG. 9 is a bottom view of an athletic helmet with the insert of FIG. 1 positioned inside the front portion of the athletic helmet.

FIG. 10 is a bottom view of a hat with the insert of FIG. 1 positioned inside the front portion of the hat.

FIG. 11 illustrates application of the insert of FIG. 1 into the front portion of an athletic helmet.

FIG. 12 illustrates application of the insert of FIG. 1 into the front portion of a hat.

FIG. 13 is a partial sectional view of the disclosed insert.

FIG. 14 is an exploded view of one embodiment of the disclosed insert.

FIG. 15 is an exploded view of one embodiment of the disclosed insert.

FIG. 16 is an exploded view of one embodiment of the disclosed insert.

FIG. 17 is a perspective view of one embodiment of the disclosed insert positioned on the suspension headband of the construction hard hat.

FIG. 18 is a top-down view of one embodiment of the disclosed insert.

FIG. 19 is a top-down view of one embodiment of the disclosed insert.

FIG. 20 is a top-down view of one embodiment of the disclosed insert.

FIG. 21 is a top-down view of one embodiment of the disclosed insert.

FIG. 22 is a top-down view of one embodiment of the disclosed insert.

FIG. 23 is a top-down view of one embodiment of the disclosed insert.

FIG. 24 is a top-down view of one embodiment of the disclosed insert.

FIG. 25 is a top-down view of one embodiment of the disclosed insert.

FIG. 26 is a top-down view of one embodiment of the disclosed insert.

FIG. 27 is a top-down view of one embodiment of the disclosed insert.

FIG. 28 is a top-down view of one embodiment of the disclosed insert.

FIG. 29 is a top-down view of one embodiment of the disclosed insert.

FIG. 30 is a top-down view of one embodiment of the disclosed insert.

FIG. 31 is a top-down view of one embodiment of the disclosed insert.

FIG. 32 is a perspective view of one embodiment of the disclosed insert.

FIG. 33 is a perspective view of one embodiment of the disclosed insert.

FIG. 34 is a back perspective view of one embodiment of the disclosed insert.

FIG. 35 is a top-down view of one embodiment of the disclosed insert.

FIG. 36 is a front view of one embodiment of the disclosed insert.

FIG. 37 is a right side perspective view of one embodiment of the disclosed insert.

FIG. 38 is a right side view of one embodiment of the disclosed insert.

FIG. 39 is a partial sectional view of the disclosed insert.

FIG. 40 is a partial sectional view of the disclosed insert.

FIG. 41 is a partial sectional view of the disclosed insert.

FIG. 42 is a partial section view of the disclosed insert.

### DETAILED DESCRIPTION

Various user interfaces and embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims. It is understood that various omissions and substitutions of equivalents are contemplated as circumstances may suggest or render expedient, but these are intended to cover application or embodiments without departing from the spirit or scope of the claims attached hereto. Also, it is to be understood that the phraseology and terminology used herein are for the purpose of description and should not be regarded as limiting.

One embodiment of this disclosure relates to a disposable insert that wicks moisture away from the user's forehead or other facial surface. The insert can be a disposable, moisture-absorbing device that is affixed to headwear, such as a helmet, hat, hard hat, goggles, or facemasks. Specifically, the insert can be affixed to athletic head coverings used in sports, such as, but not limited to, golf, motocross, cycling, baseball, football, hockey, and lacrosse. For example, the insert can attach to the portion of the headwear that is normally in contact with the wearer's forehead and/or temples. In another example, the insert can attach to a chinstrap of athletic headwear. Alternatively, the insert can attach itself to a user's head using other means that do not require a head covering. The disposable, absorbent insert is specifically designed to fit within the aforementioned headwear and other related head coverings.

In one embodiment, the material from which the disposable insert is comprised is not elastic. In another embodiment, at least some of the material from which the disposable insert is comprised is elastic. For example, the absorbent portion of the insert can be made from a non-elastic material, whereas the fabric or foam that covers the absorbent part can be made from elastic material. In another example, both the absorbent portions and the fabric or foam cover can be made from, or include, elastic material. In some embodiments, the insert is made using materials similarly used in the making of feminine hygiene pads. The insert can, in some embodiments, be recyclable or made of biodegradable materials. Additionally, the materials used in the insert can be breathable so the insert does not block ventilation from the head covering. In some embodiments, any of the layers can incorporate flame-resistant fibers, as described further below.

The disposable absorbent insert can be adhered to the curved inside surface of the headwear. The insert can be easily attached to the headwear immediately before activity and can then be removed from the headwear and discarded after physical activity has ceased. In addition, a soiled insert may be replaced with a fresh insert during activities (e.g., during a break, during a timeout, between periods, at halftime, etc.).

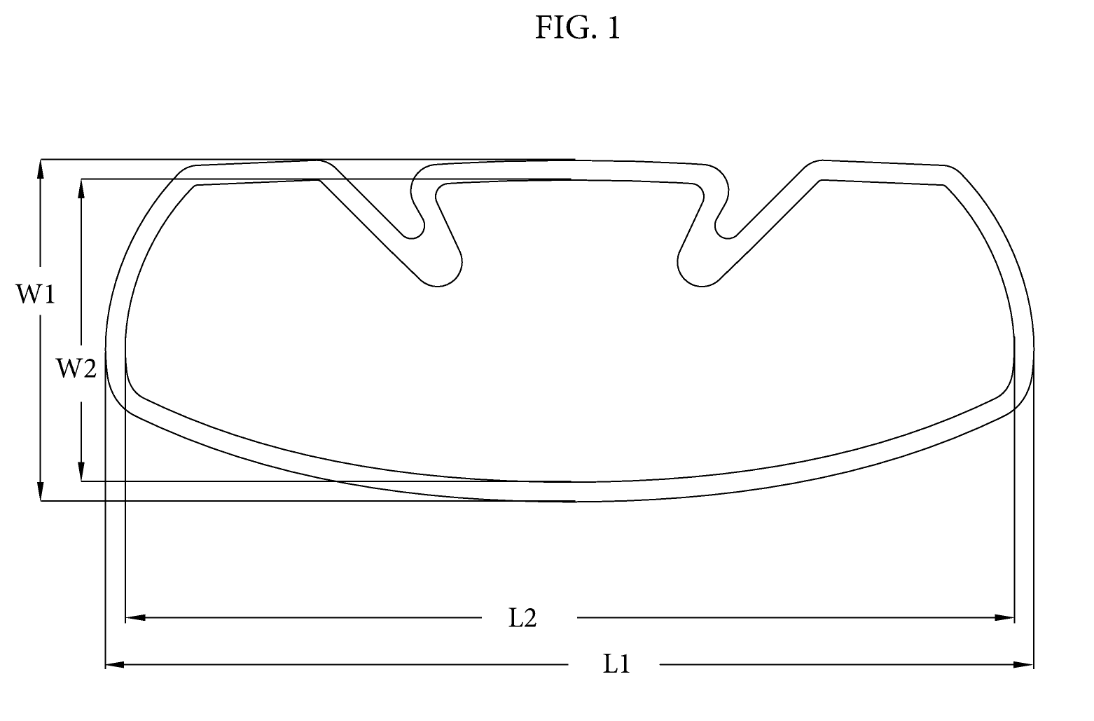

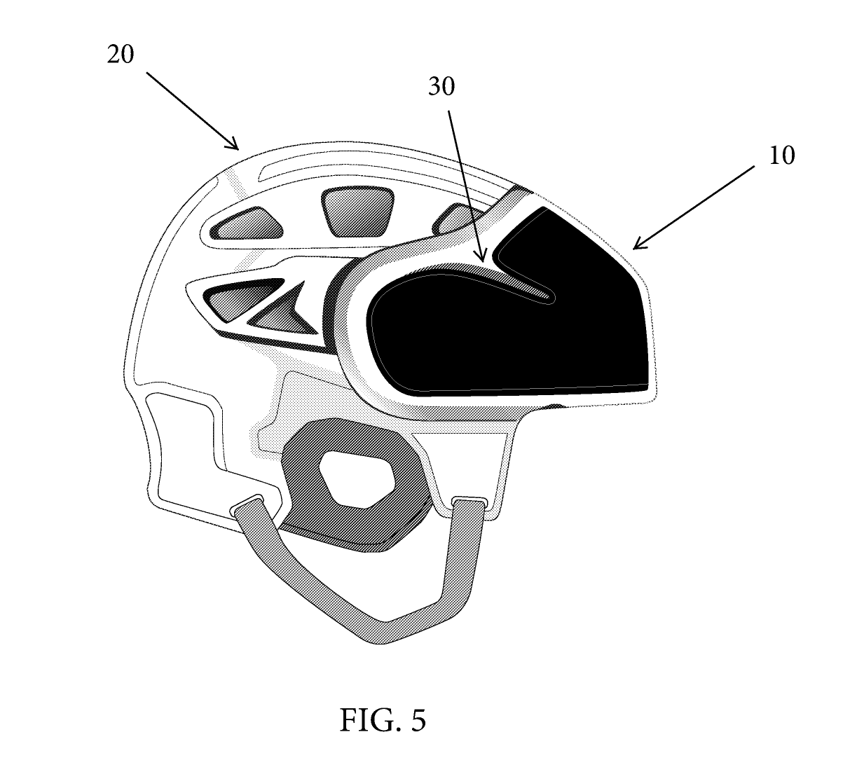

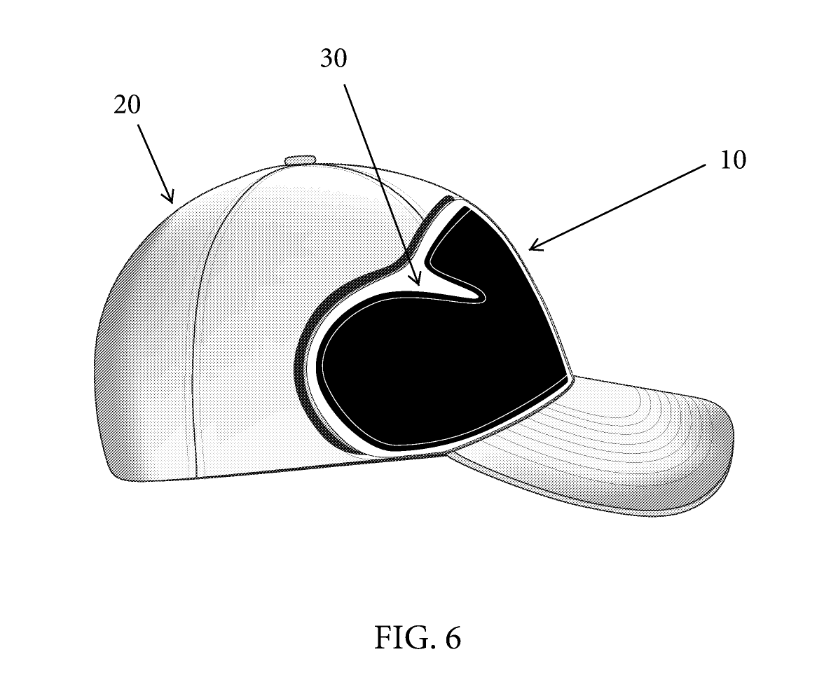

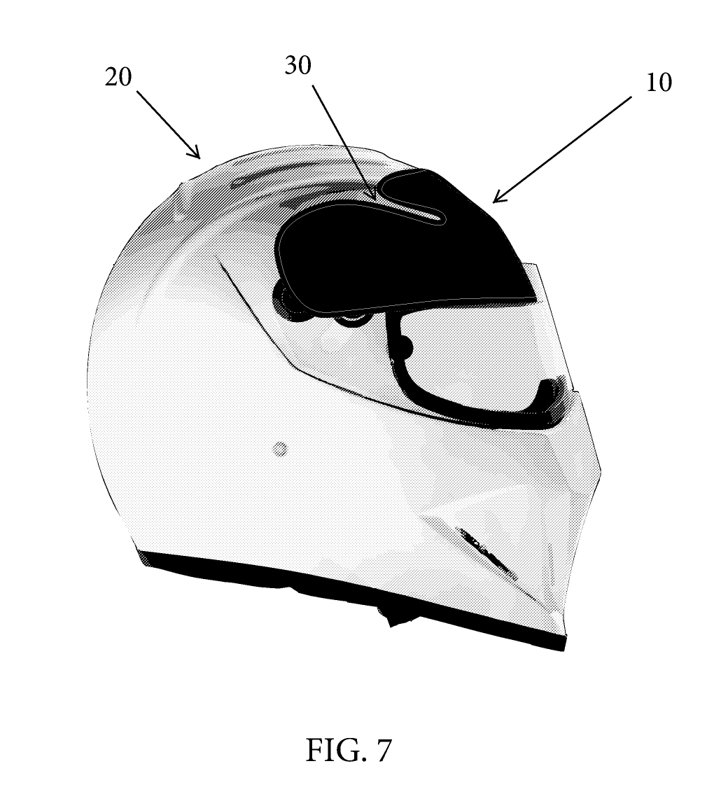

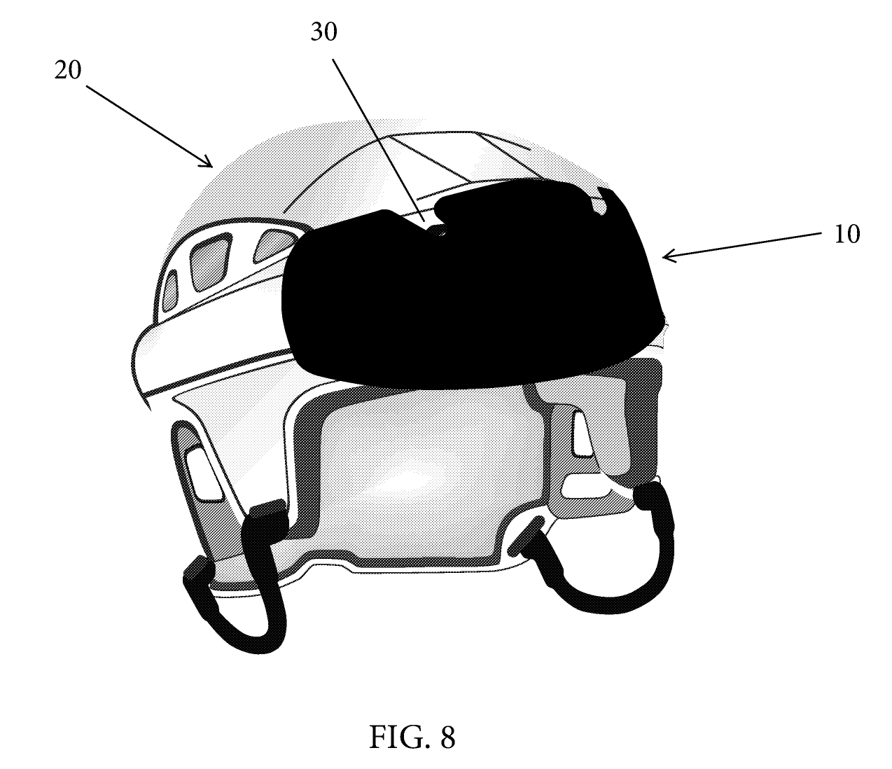

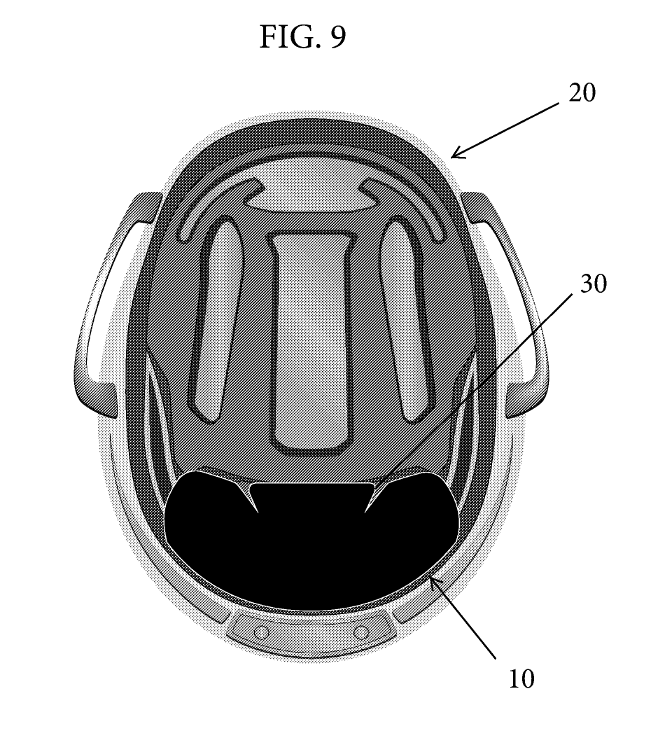

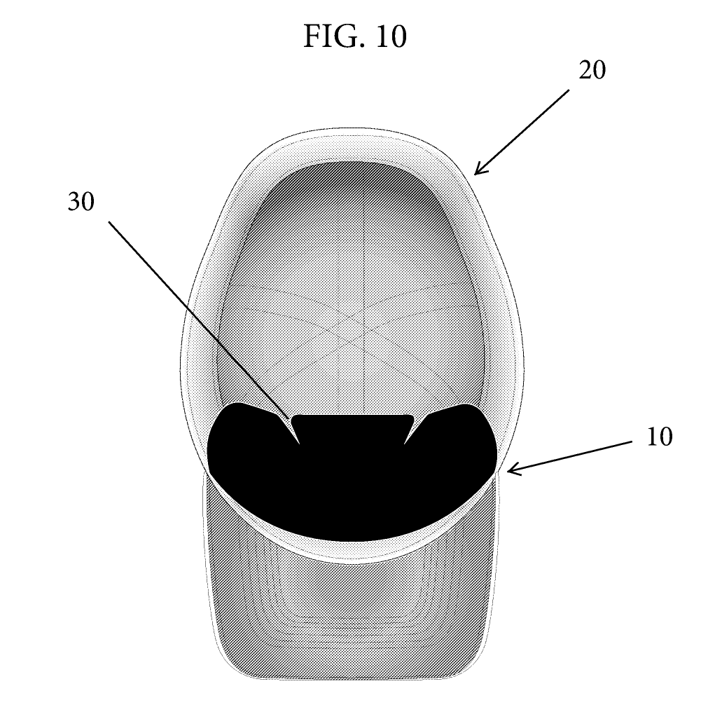

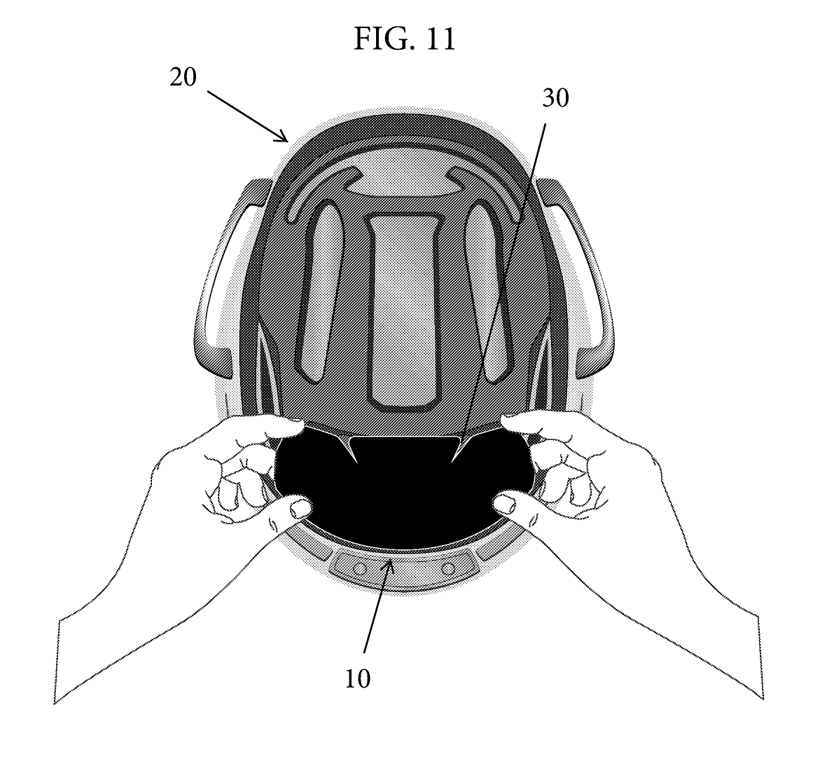

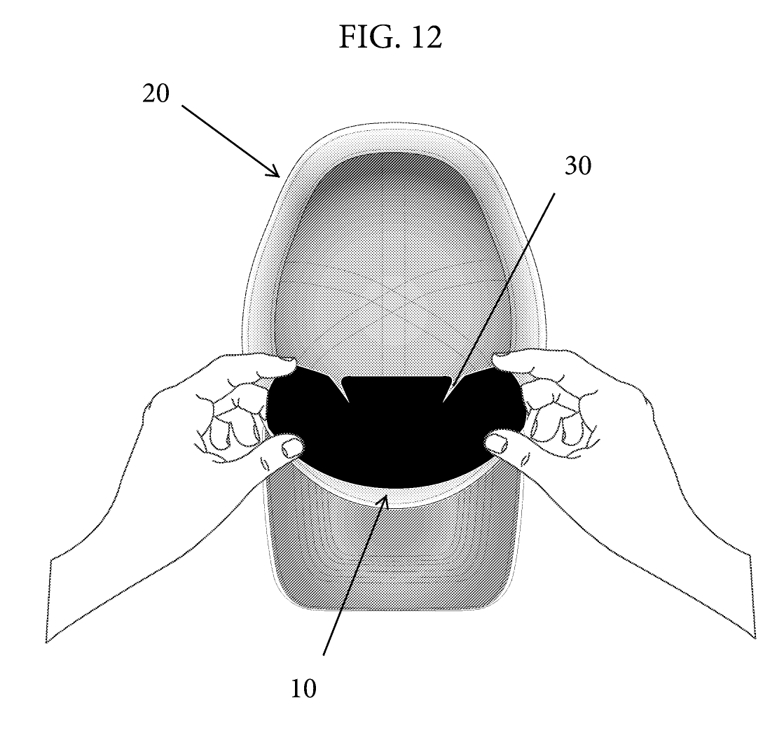

One embodiment of a device for absorbing and retaining sweat is an insert, as illustrated in FIGS. 1-31. FIGS. 1-14 illustrates various embodiments of an insert 10, wherein the insert 10 is intended to attach directly to the inside surface of headwear 20. FIGS. 1-4 are a view of one embodiment of an insert 10, wherein the insert 10 is laying flat. FIGS. 5-7 are side views of various types of headwear 20 (for example, a hockey helmet, hat, hard hat, welding helmet, and motor sport or racing helmet), illustrating the orientation of the insert 10 within the headwear 20, from the side. FIG. 8 is a perspective view of the headwear 20 illustrating the orientation of the insert 10 within the headwear 20, when the insert attaches directly to the headwear 20. FIGS. 9 and 10 are bottom views of the headwear 20 illustrating the orientation of the insert 10 within the headwear 20. FIGS. 11 and 12 illustrate one example of how a user can attached the insert 10 into headwear 20.

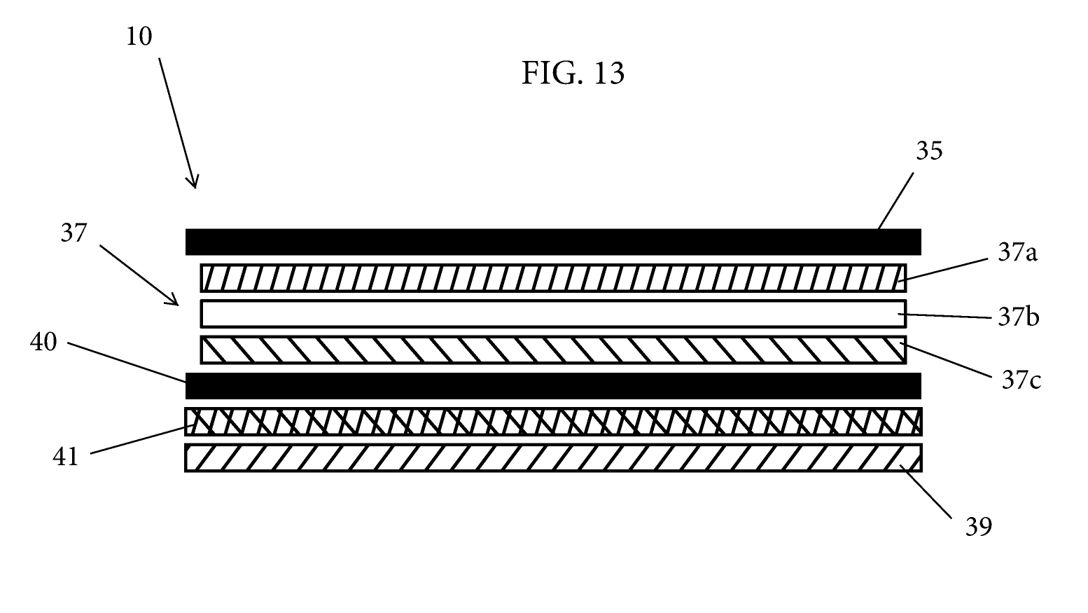

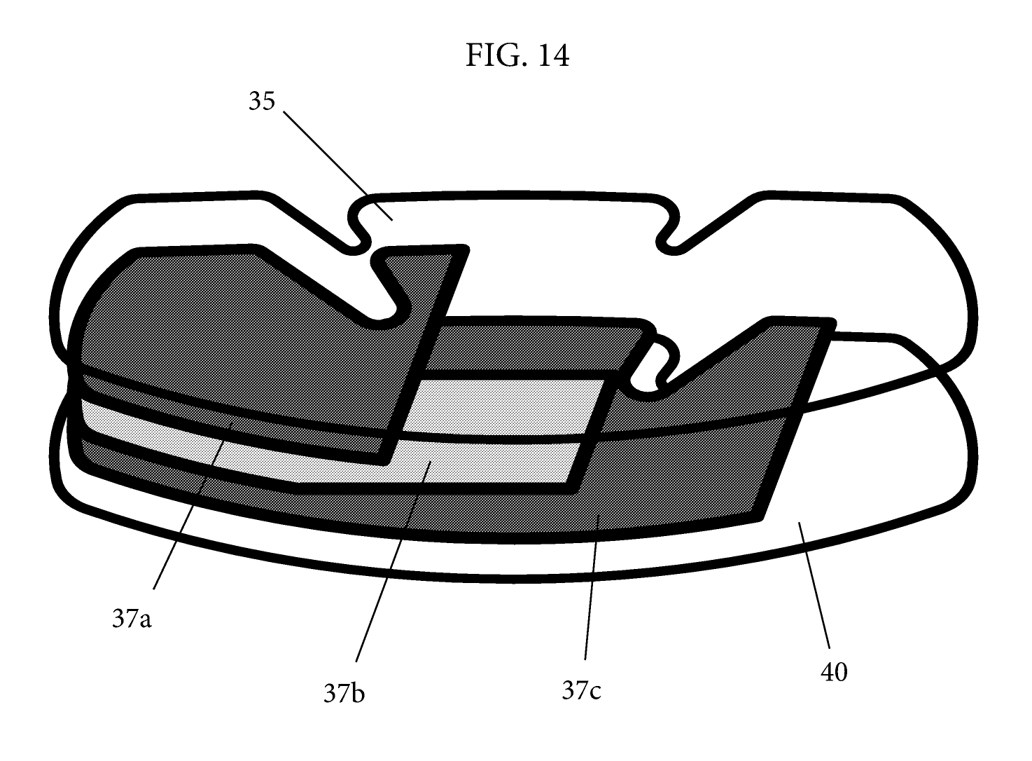

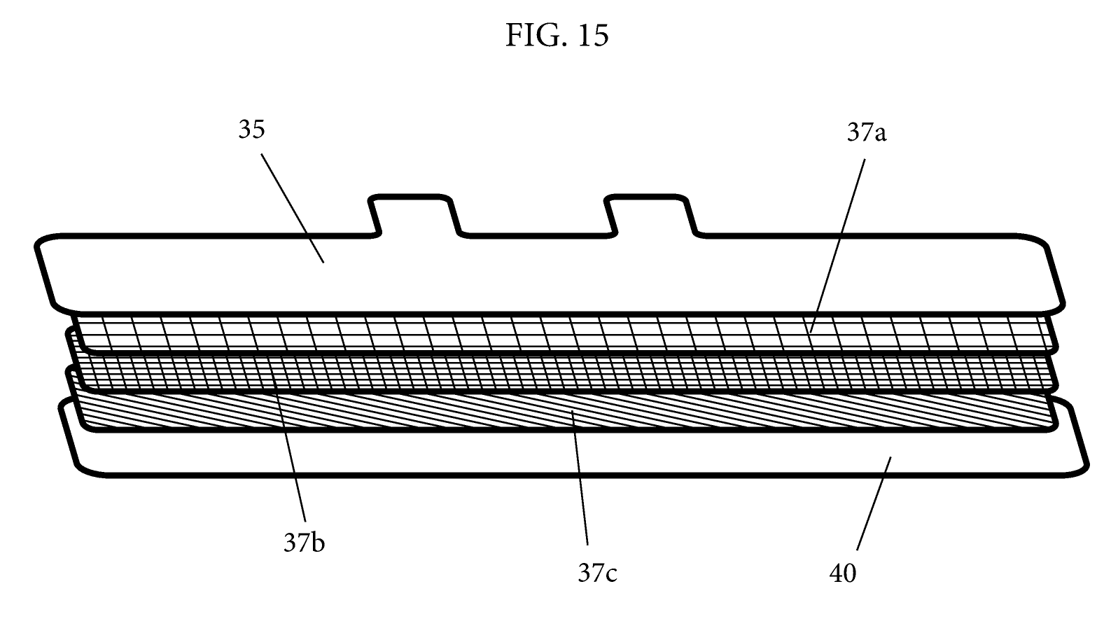

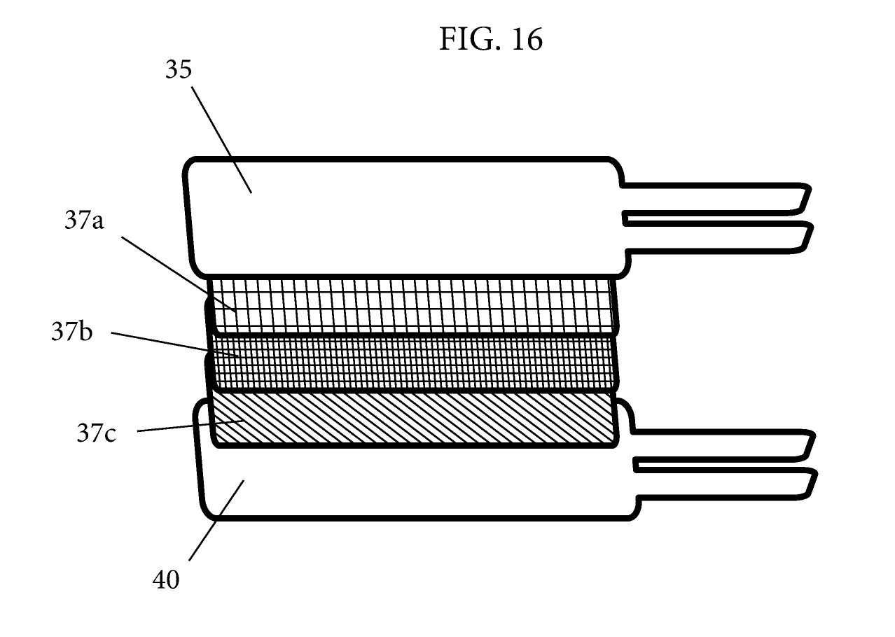

FIG. 13 is a partial sectional view of an insert 10 illustrating the layers of the insert 10. FIGS. 14-16 are exploded views of the layers of the insert 10 according to different embodiments. For example, FIG. 14 illustrates an embodiment that can be attached directly to the inside of headwear 20. FIGS. 15 and 16 illustrate embodiments that can be attached to, for example, the forehead-contacting surface of a headband portion of a headwear suspension system, such as a construction hard hat, welding helmet, bump cap, visor, or protective headwear (hereinafter referred to as a “suspension headband”).

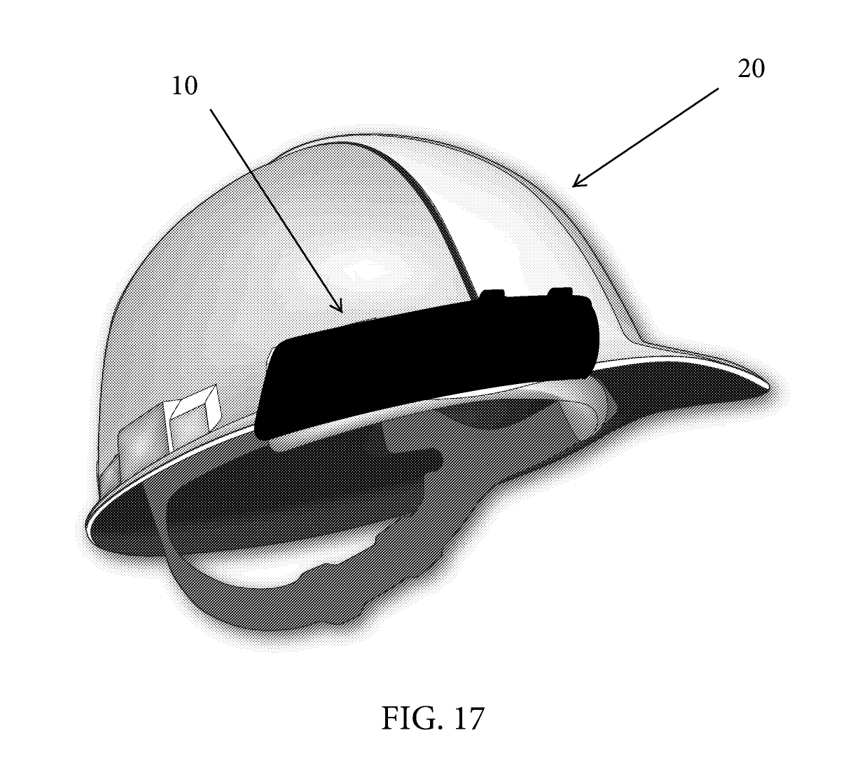

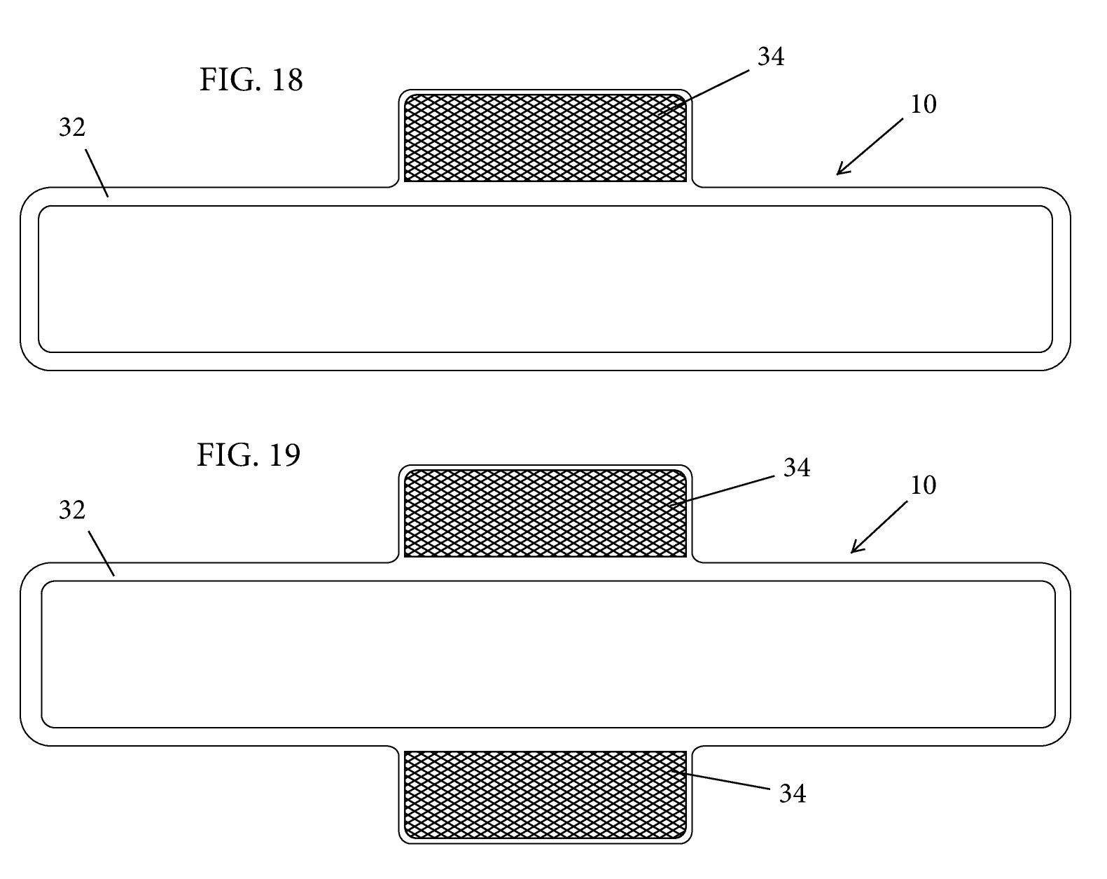

FIGS. 15-31 illustrate various embodiments of an insert 10, wherein the insert 10 is intended to attach to a suspension headband, such as a construction hard hat suspension headband. Generally, the insert 10, when intended to attach to a suspension headband, has a rectangular main body and can have at least one tab to wrap around the edge of the suspension headband and secure the insert 10 in place. FIG. 17 is a perspective view of the headwear 20 illustrating the orientation of the insert 10 within the headwear 20, when the insert 10 attaches to the suspension headband of a construction hard hat. FIGS. 18-31 illustrate various attachment configurations for the insert 10.

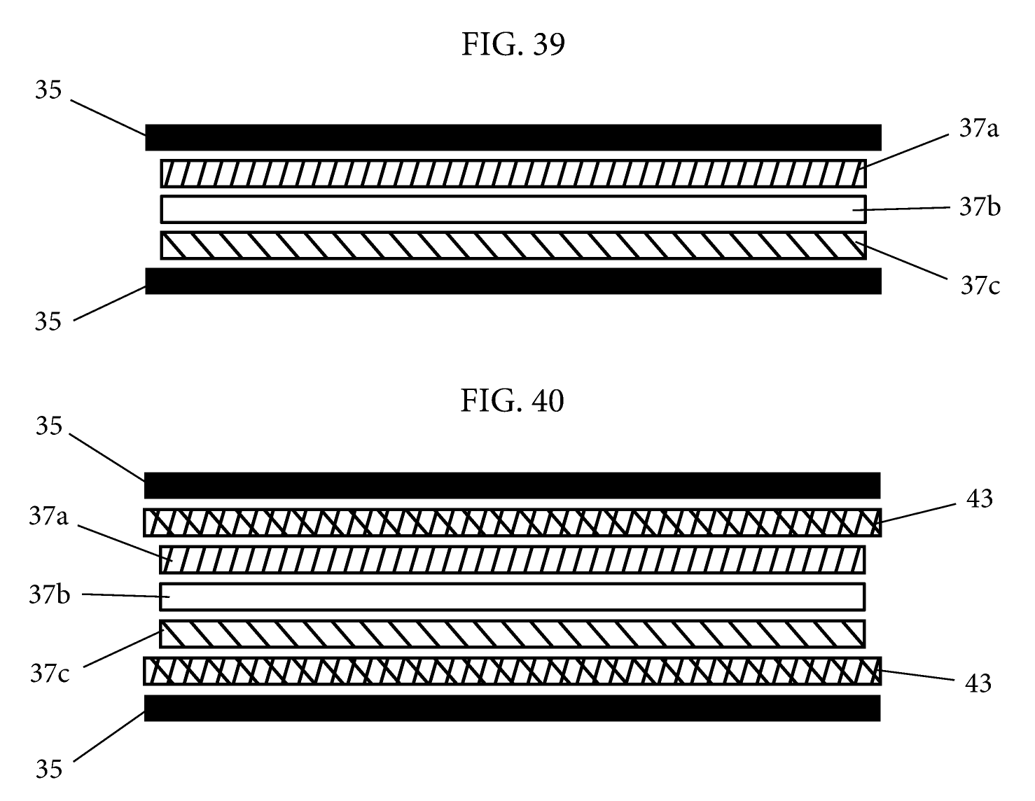

This device for absorbing and retaining sweat can, in one embodiment, be fitted against the inside surface of an athletic helmet or hat or a construction hard hat. The insert, when placed on the front portion of the helmet, hat, or hard hat, functions to wick moisture away from the head of the user and prevent moisture from dripping down into the eyes, nose, mouth and overall facial area of the user. If placed at the back of a helmet, hat, or hard hat, it can also prevent moisture from dripping down onto the neck of a user. The insert 10 can be in direct contact with the user's forehead and can be formed of an absorbent material. In one embodiment, as illustrated in FIGS. 14-16, the insert 10 is comprised of several layers. These layers function to wick moisture away from the user's forehead, trap moisture inside the disposable insert, and prevent moisture from dripping down onto the face of the user. In another embodiment, the device for absorbing and retaining sweat can be a band 50, such as a headband or sweatband, that secures around a user's head. In this embodiment, the band 50 is comprised of several layers, as illustrated in FIGS. 39-41.

The device for absorbing and retaining sweat can benefit its user in many ways. The most apparent benefit is keeping sweat originating on the user's forehead and hair from dripping down onto the user's face or neck. When perspiration drips down onto a user's face, it can impair the user's vision. This impairment of vision, in the case of athletes, can be a painful annoyance and can disrupt the play and overall performance of the athlete during the run of play. In the case of construction workers or racecar drivers, impaired vision can result in distractions and severe injury.

In some activities (for example, hockey, motocross, cycling, golf, construction, or military tactical activities), many individuals use glass/plastic facial shields, goggles, glasses, or other eyewear, which have a high tendency to fog up during use due to perspiration and moisture entering into the area between the user's face and the glass or plastic barrier. The disposable insert of this disclosure can help its user by absorbing the sweat and eliminating almost all perspiration from dripping down onto the user's face or face covering. This will greatly decrease the probability of the glass/plastic barrier fogging up and will not impair the user's vision during the activity. This enables hockey players, for example, to compete at a high level without the distraction of a fogged facial shield. For construction workers or military men and women, this reduces a safety risk associated with a fogged facial shield.

Additionally, headwear, such as a helmet or hard hat, is usually unable to be thoroughly washed and, therefore, will lock moisture, oils, and odors in. Use of the insert enables the sweat, oils, and odors to get locked in the insert and, therefore, reduces scents odors from accumulating in the head covering.

Skin hygiene is another important benefit from using the insert 10 or band 50. Many adult and teenage athletes who wear helmets or sweatbands during their sporting activities experience increased skin irritation and facial acne during participation while in season. Construction workers may be subject to skin irritation throughout every season, especially the hot, summer months. Sweat and oil from the user's hair and forehead can drip down onto the face, which can clog facial pores and cause unwanted facial blemishes. Also, the pressure and direct contact that a user's forehead has with the helmet or hard hat can cause skin rashes and increased acne as well. The disposable device can prevent sweat and oil from dripping down onto the face and clogging the user's facial pores. The soft material of which the disposable device is made can provide a clean and comfortable barrier between the user's forehead and helmet or hard hat, when using the insert 10, and can eliminate skin irritation and blemishes caused by the friction between the forehead and the inside surface of the helmet or hard hat. In some embodiments, the insert 10 or band 50 may have an FDA-approved skin-contacting material and/or may contain acne cream, other acne topicals, or an antibacterial or antimicrobial. The insert 10 or band 50 could also contain lotion, aloe, vitamins, pharmaceuticals, or bug spray and can be hypoallergenic, latex free, or both. Further, as described elsewhere in the disclosure, the insert 10 or band 50 may be flame resistant.

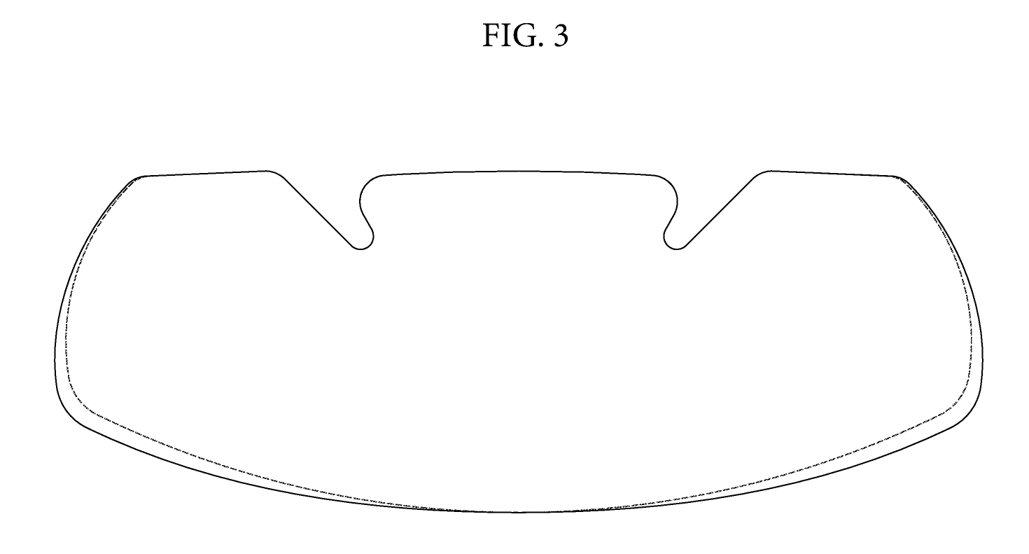

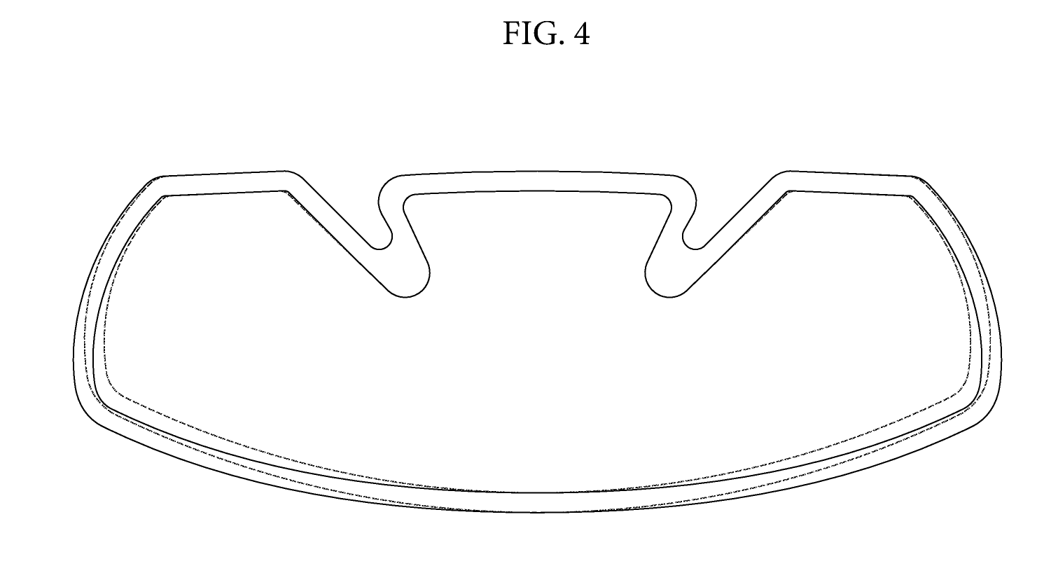

In one embodiment, illustrated in FIG. 1, a length L1 of the insert 10 is between 8 and 9 inches (ex: 8.8379 inches) and a width W1 of the insert is between 3 and 4 inches (ex: 3.25 inches). In another embodiment, the disposable insert can have the dimensions of: Length L: 180 mm to 330 mm (e.g., length=250 mm); Width W: about 100 mm (e.g., width=97 mm); Thickness T: about 2 mm to about 10 mm (or even thinner, from about 2-7 mm (e.g., thickness=4-5 mm)). FIGS. 3 and 4 illustrate variations in size and shape that the liner 10 may have. FIG. 3 illustrates the outer border variations and FIG. 4 illustrates variations in the inner and outer portions of the liner according to two different sizes, wherein a preferred embodiment is slightly wider and has a smaller angle along a lower curved edge 22. For example, the corner radius of the lower portion of rounded end edges 26, 28 can be 0.50 inches but is preferably 0.51 inches.

In one embodiment, illustrated in FIG. 31, the insert 10 is used in the suspension headband of headwear 20, such as a helmet or hard hat, and has length L3, width of the main body W3, width of the main body and tabs W4, and tab gap width G1. Length L3 can be between 10 and 11 inches (for example, 10.375 inches). Width W3 can be approximately two inches and width W4 can be approximately three inches. Gap width G1 can be between one and two inches (for example, 1.60 inches) and the width of each tab can be between 0.5 and 1.0 inches (for example, 0.80 inches).

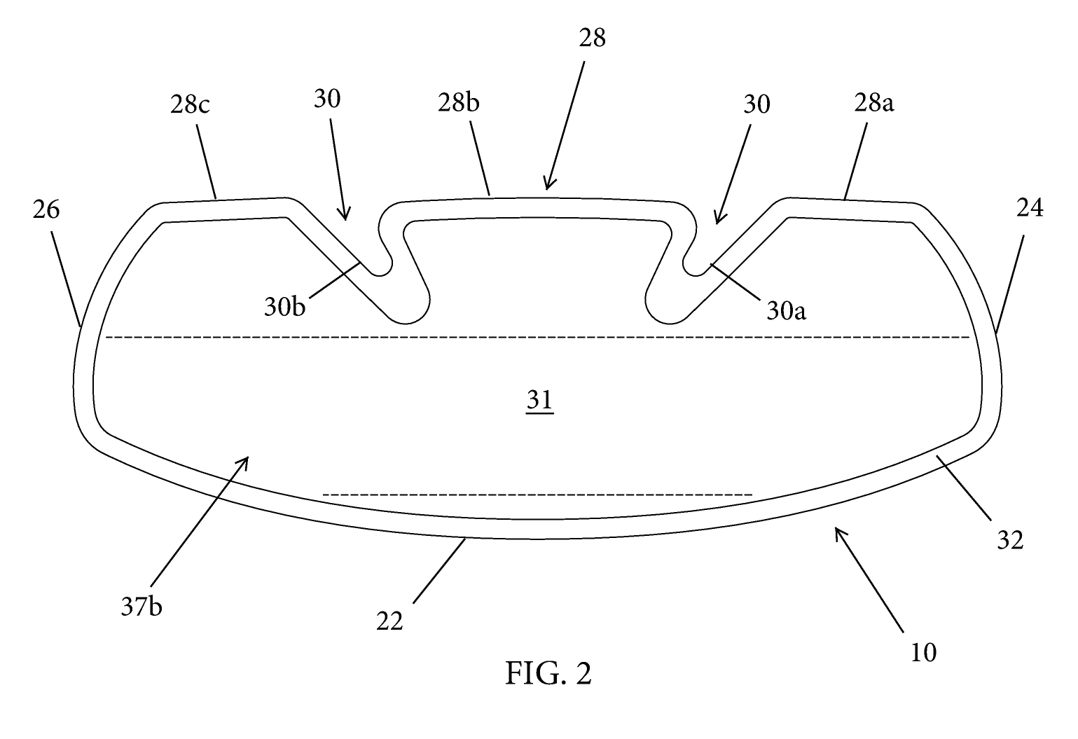

In one embodiment, as illustrated in FIG. 2, each insert 10 is generally uniform in thickness and has a lower curved edge 22 and rounded end edges 24 and 26. In some embodiments, the main body of the insert 10 is uniform in thickness while the edges of the insert 10 form a seal that has a thinner thickness than the main body. Each insert 10 has a top edge 28, which is interrupted by one or more slits 30, thereby creating gaps or grooves 30a, 30b, which are cut through all layers of the insert and angled towards a central portion of the insert. In one embodiment, two gaps 30a, 30b are provided, and they are angled inwardly toward a central portion 31 of insert 10. For example, the gaps 30a, 30b may extend from 40-50 degree angles from the top edge 28 toward a central portion 31, and the gaps 30a, 30b may be broader near top edge 28 than the central portion 31. In another example, the corner radius of the gaps 30a, 30b near the central portion 31 is 0.13 inches.

The gaps 30a, 30b allow insert 10 to naturally follow the contours of the inside curved surface of headwear 20. Moreover, such angling of the gaps 30a, 30b allows the gaps to lie naturally along headwear 20 contour lines, such as front-to-back lines shown in FIGS. 5-7. Accordingly, the gaps 30a, 30b caused by slits 30 are least obtrusive to the user. The top edge 28 is thus divided into outer top edge portions 28a, and 28c and inner top edge portion 28b. Top edge portions 28a and 28c are generally curved, while top edge portion 28b is generally linear. However, in some embodiments, top edge portions 28a, 28b, and 28c can be generally linear or collinear. The corners of the inner top edge portion 28b that contact the gaps 30a, 30b can each have a corner radius of 0.27 inches.

In one embodiment, as illustrated in FIGS. 18, 24, and 25, each insert 10 is configured to attach to the inside of a hat, helmet, or a hard hat suspension headband, is generally uniform in thickness, has a rectangular main body, and at least one tab. However, as illustrated in FIG. 30, some embodiments of the insert 10 that are configured for attachment to the inside of a helmet or hard hat suspension headband do not have a tab. The tab can be an adhesive tab 34, a hook and loop tab 36, or a snap/button tab 38.

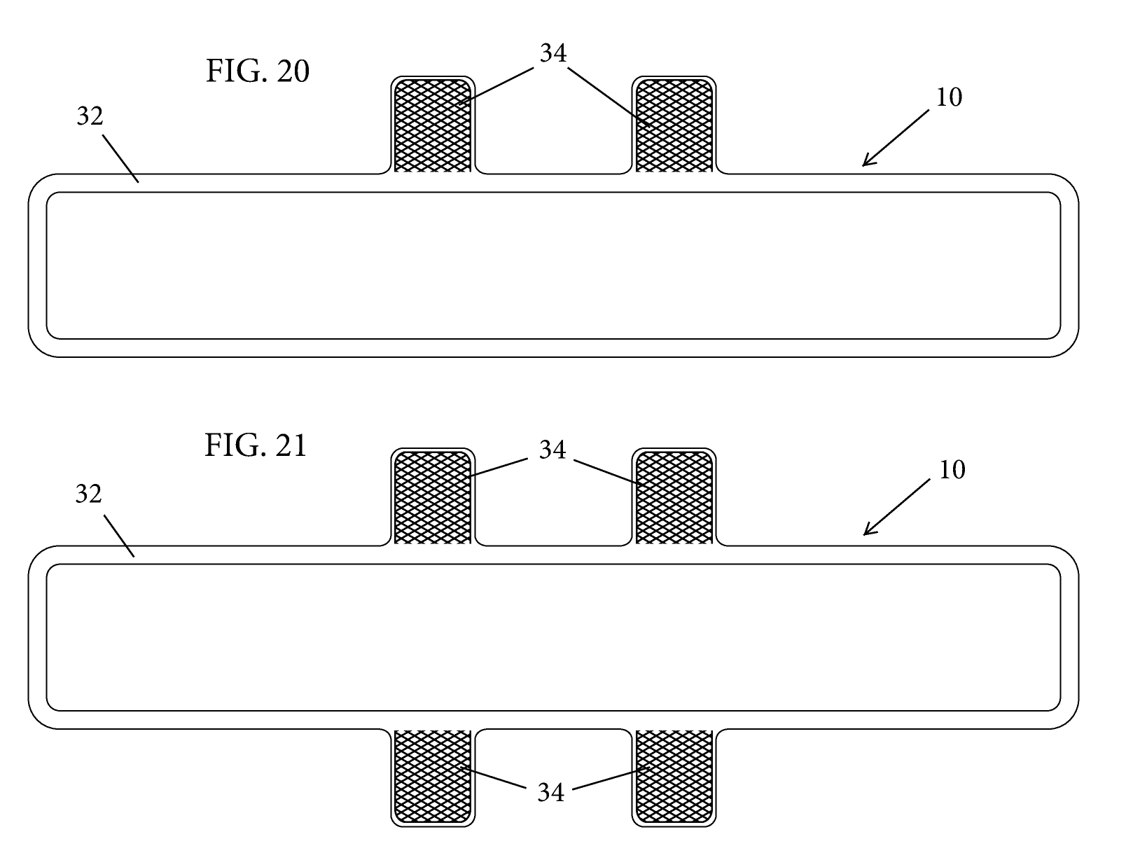

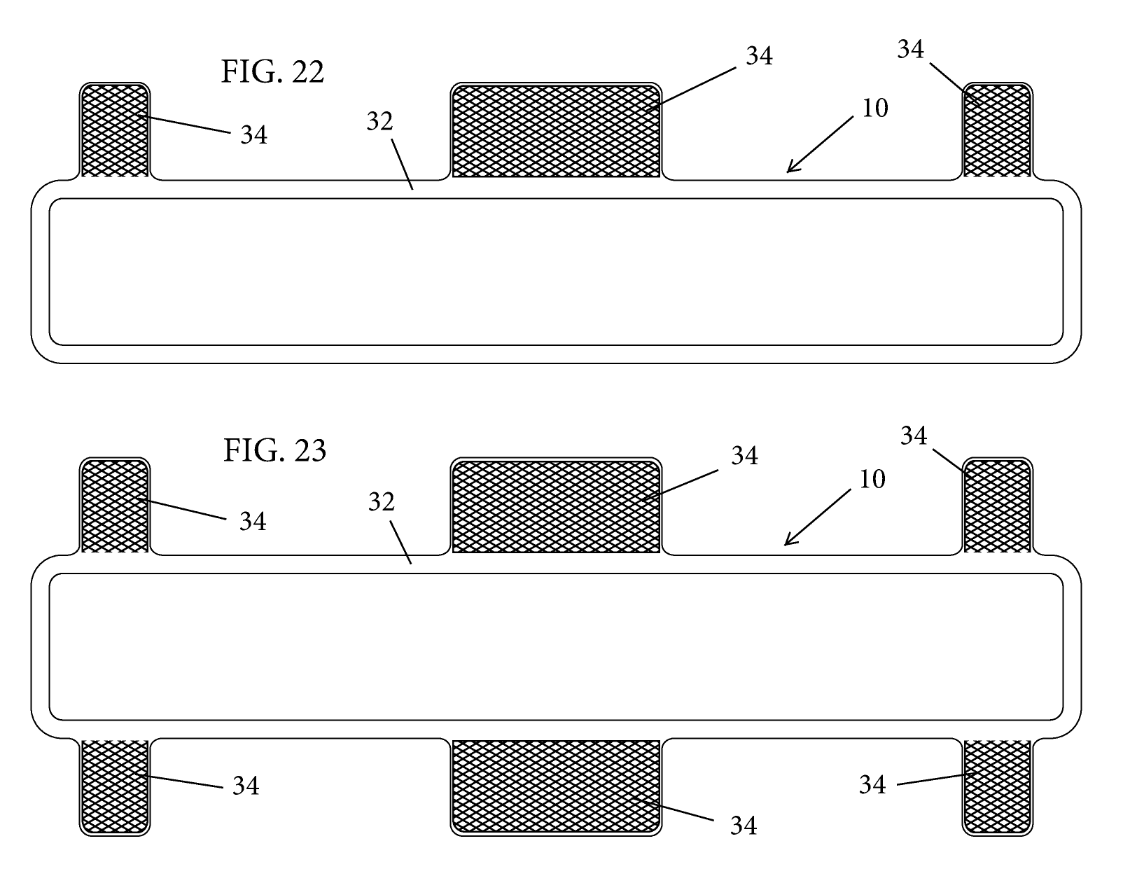

The tab(s) 34, 36, 38 may have any variety of configuration, but preferably protrude from the top or bottom edge of the rectangular main body of the insert. For example, the insert 10 may have one long tab centered on the longer portion of the rectangular main body, as illustrated in FIGS. 18 and 24-25. In another example, the insert 10 may have two long tabs, wherein a first tab is centered on the top edge of the longer portion of the rectangular body and a second tab is centered on the bottom edge of the longer portion of the rectangular body, as illustrated in FIG. 19. In a further example, the insert 10 may have a plurality of short tabs that are evenly centered around the middle of the longer portion of the body, wherein the short tabs are located on the top edge of the rectangular body, as illustrated in FIG. 20 or the top and bottom edges of the rectangular body, as illustrated in FIGS. 21 and 26-27. In another example, the insert 10 may have a combination of long and short tabs along the top and bottom edges of the rectangular body. For example, as illustrated in FIG. 22, the insert 10 may have short tabs on the ends of the top edge of the longer portion of the rectangular body and a long tab centered on the top edge of the longer portion of the rectangular body. As illustrated in FIGS. 23 and 28-29, the insert 10 may have short tabs on the ends of the top and bottom edges of the longer portion of the rectangular body and long tabs centered on the top and bottom edges of the longer portion of the rectangular body.

Any of the configurations described above are conducive to adhesive tabs 34, wherein the adhesive tabs 34 fold over the edge of the suspension headband and attach to the opposite side of the suspension headband. For example, the embodiments with tabs on the top edge of the rectangular body can fold over the top edge of the suspension headband and embodiments with tabs on the top and bottom edges of the rectangular body can fold over the top and bottom edges of the suspension headband, respectively.

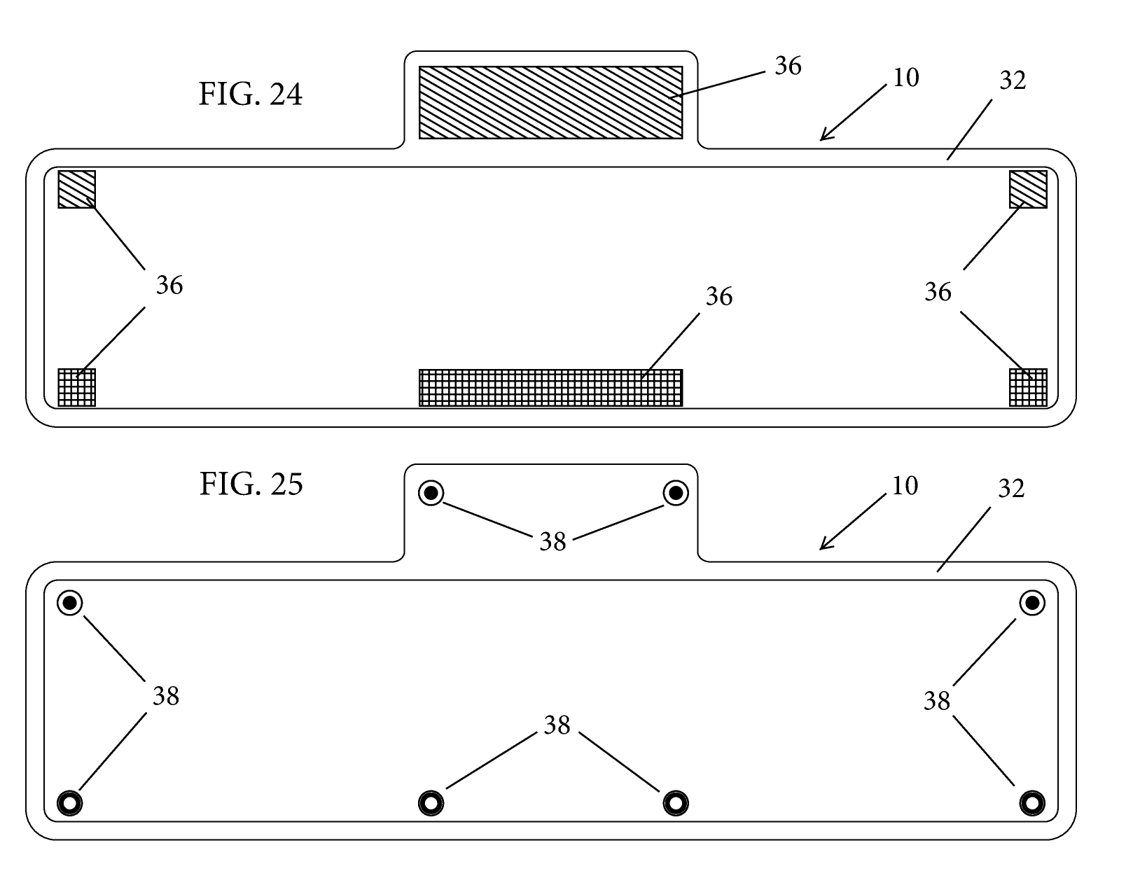

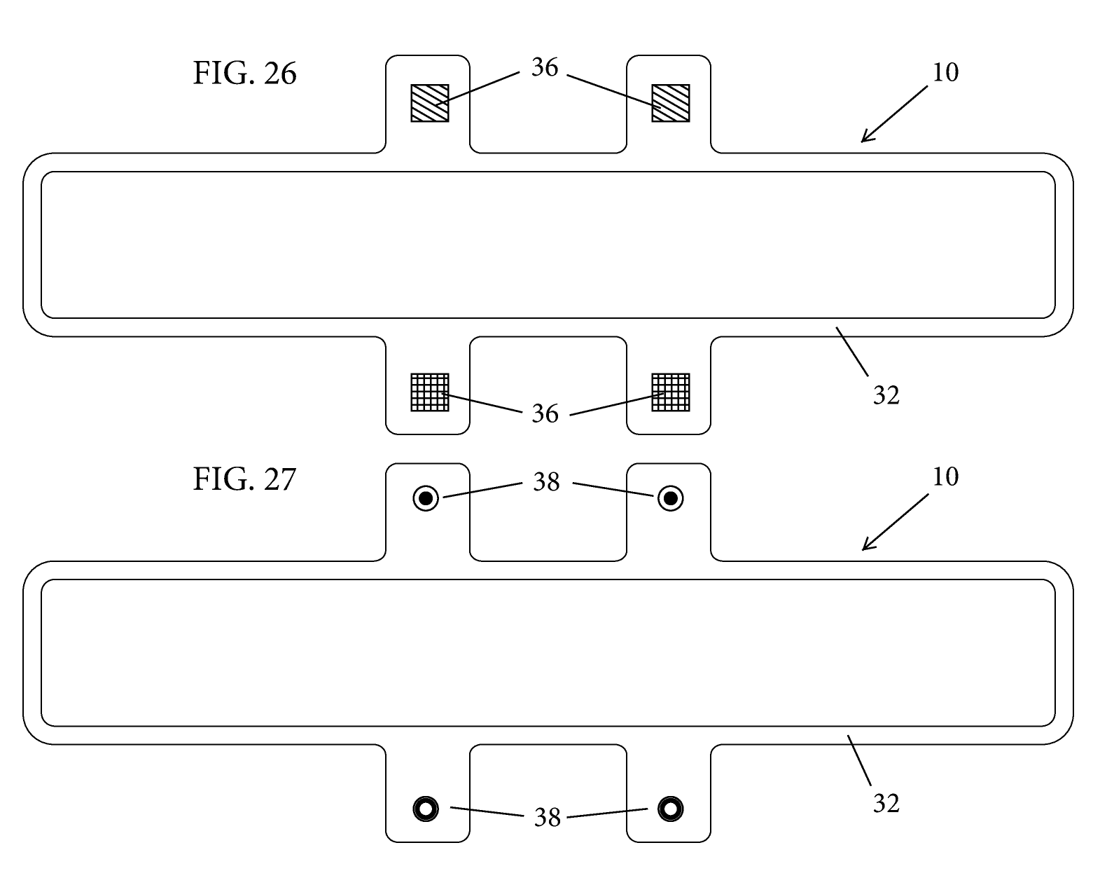

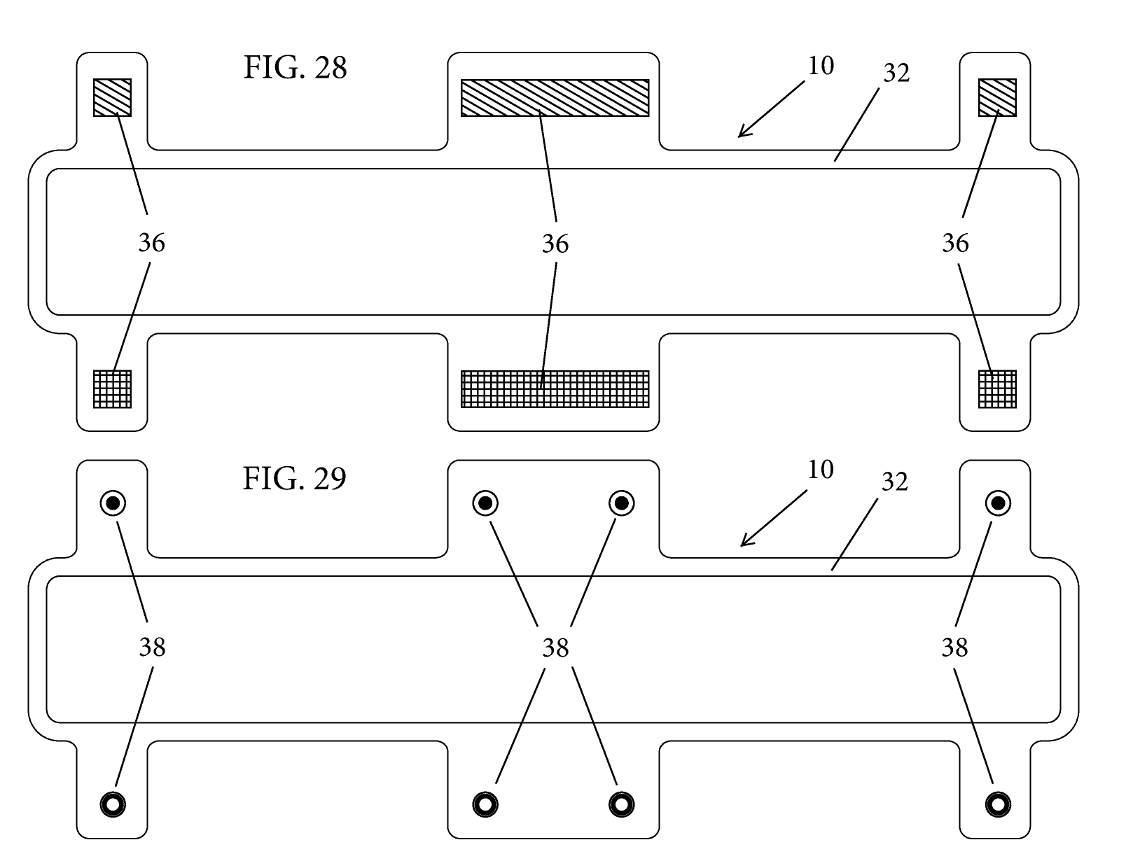

The configurations illustrated in FIGS. 24, 26, and 28 are conducive to hook and loop tabs 36, wherein the tab along the top part of the rectangular body can fold over the suspension headband and either attach to a hook and loop portion on the rectangular body itself, as illustrated in FIG. 24, or to another tab along the bottom part of the rectangular body, as illustrated in FIGS. 26 and 28. In some embodiments of the hook and loop configurations, the rectangular body can have and adhesive layer 41 that also attaches to the suspension headband.

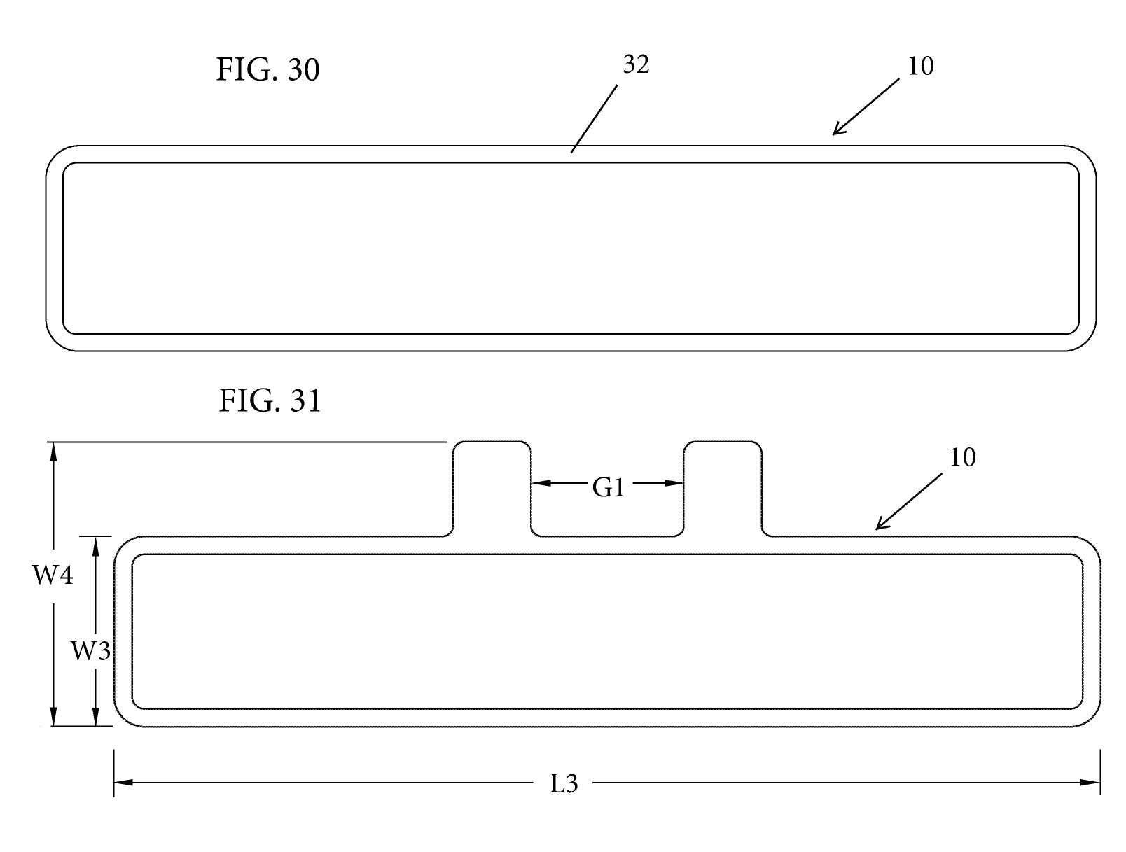

The configurations illustrated in FIGS. 25, 27, and 29 are conducive to snap/button tabs 38, wherein the tab along the top part of the rectangular body can fold over the suspension headband and either attach to a snap/button portion on the rectangular body itself, as illustrated in FIG. 25, or to another tab along the bottom part of the rectangular body, as illustrated in FIGS. 27 and 29. In some embodiments of the snap/button configurations, the rectangular body can have and adhesive layer 41 that also attaches to the suspension headband.

Regardless of the headwear 20 to which the insert 10 attaches, the insert 10 may be multilayer in construction. A top, moisture wicking layer 35, illustrated in FIGS. 14-16, contacts the user, is preferably moisture wicking, and may have a moisture-absorbent functional attribute to draw sweat away from the user. The moisture wicking layer 35 can be made of a hydrophilic material that functions to wick moisture from the user's skin surface and transfer it into the core of the product. The top, moisture wicking layer 35 may have an embossed or printed pattern or logo. An inner layer, or core, is moisture absorbent and may have moisture retention attributes.

In some embodiments, the core may be formed from one layer of moisture absorbent/retentive material. Alternatively, it may be formed from two or more layers of such material, as illustrated in FIGS. 13-16. These moisture absorbent/retentive layers form the core of the insert 10 and may be formed from non-woven materials including, for example, non-woven fluff, non-woven wood, wood pulp, wood pulp fibers, cotton, rayon, air laid, paper or non-woven absorbent polymeric fibers, other cellulosic materials, or the like. For instance, in one example, the core includes two layers that are moisture absorbent/retentive layers. The first layer can be made of a perforated non-woven material (e.g., spunlace) material, and the second layer can be an absorbent paper.

In another embodiment, as illustrated in FIG. 14, the core can contain three layers, wherein the first layer is a first, absorbent airlaid layer 37a, the second layer is a super absorbent polymer (SAP) layer 37b, and the third layer is a second, absorbent airlaid layer 37c. As illustrated in FIG. 2, the second, super absorbent core layer 37b may be generally rectangular in shape, while the first and second absorbent airlaid layers 37a, 37c take the general shape of the insert 10. A fluid or liquid impervious layer 40 may also be provided (e.g., a polyethylene film) underneath the core to prevent fluid from contacting the user's forehead. In one embodiment, the type of materials used for feminine hygiene pads may suffice for the core. Therefore, the insert 10, in a preferred embodiment, has a top, moisture wicking layer 35, an absorbent core comprised of two, absorbent airlaid layers 37a, 37c that sandwich a SAP layer 37b, and a liquid impervious layer 40.

In another embodiment, a fifth, adhesive layer 41 can attach to the liquid impervious layer 40. In this embodiment, the user can attach the disposable insert 10 to headwear 20, such as a helmet, hat, or hard hat. The adhesive layer 41 can be two-sided tape, wherein one side faces the liquid impervious layer 40 and the other side faces a sixth layer that is a release liner 39 that is removed before use. The adhesive layer 41 can also be hook and loop fabric. The adhesive layer 41 can be a full coverage adhesive layer, wherein it covers the entire surface of the liquid impervious layer 40, or it can be a partial coverage adhesive layer 41, wherein it covers only a portion of the liquid impervious layer 40. In some embodiments, however, there is no adhesive layer 41.

In some embodiments, the insert 10 can further include a foam material that is moisture wicking and that completely surrounds the absorbent core 37. The foam material, in some embodiments, can be porous. The moisture wicking foam layer can, in some embodiments, be poured, filled, or wrapped around the absorbent core 37. Alternatively, the absorbent core 37 may be made of an absorbent foam material, wherein the absorbent core 37 is one absorbent foam layer entirely or is two or more absorbent foam layers. Alternatively, the moisture wicking foam can be a layer of foam, wherein one foam layer is included above the absorbent core 37 and a second foam layer is included below the absorbent core 37, thereby sandwiching the core 37 in moisture wicking foam. Similar to the layers described above, the second foam layer located below the absorbent core 37 can also be attached to an adhesive layer 41. The adhesive layer 41 can, therefore, attach to the headwear 20, such as a hat, helmet, or hard hat. Alternatively, the insert 10 having first and second layers may be configured to form a band 50. Therefore, the insert 10 can be attached directly to the user's head by wrapping around the head and securing either by pressure (for example, if the band 50 is at least partially elastic) or by attachment points on the band 50.

In some embodiments, a sealed perimeter 32 can be made when the perimeter of at least the top, moisture wicking layer 35, core layers 37, and liquid impervious layer 40 are sealed together, wherein the insert 10 has a continuous seal along the entire perimeter. In some embodiments, a sealed perimeter 32 can be made when the perimeter of at least the top, moisture wicking layer 35 and the liquid impervious layer 40 are sealed together, as illustrated in FIGS. 2 and 18-30, wherein the insert 10 has a continuous seal along the entire perimeter. In some embodiments the type of material that is sealed is polypropylene, polyethylene, hydrophilic nonwoven, or other nonwoven material. The sealed perimeter 32 can be 1-3 mm thick and can be created using means such as, but not limited to, construction glue and pressure, heating element seals (ex: hot bar, continuous, impulse), ultrasonic weld/seal/staking, material welding and bonding, sewing, transfer adhesives and pressure (ex: hot melts, construction adhesives, or transfer adhesives and tapes), high frequency welding, thermoplastic staking, infrared staking, radio frequency welding, laser welding, heat and pressure, or head seal coating. In a preferred embodiment, the sealed perimeter 32 is created using an impulse heating element seal. Sealing these layers together enables moisture to be locked in the core 37. In one embodiment, the sealed inner portion has a length L2 of between 8 and 9 inches (ex: 8.4629 inches) and a width W2 of between 2 and 3 inches (ex: 2.875 inches), as illustrated in FIG. 1.

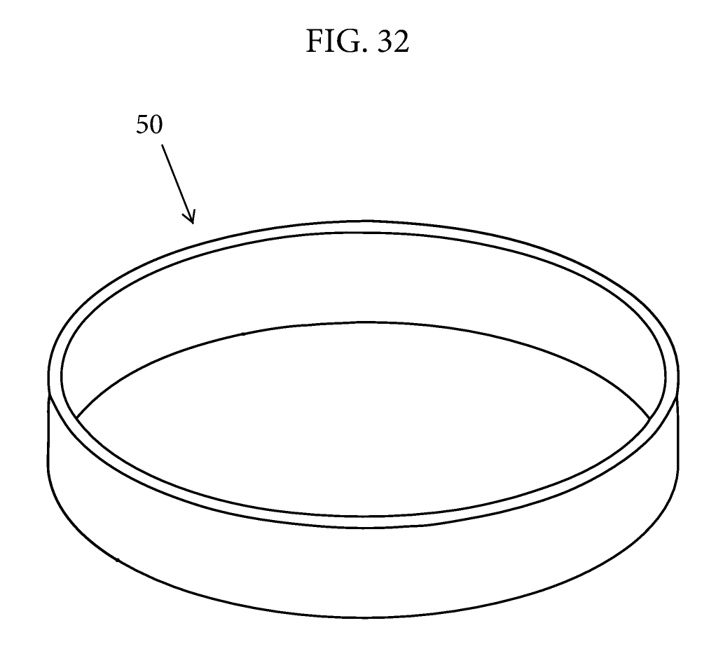

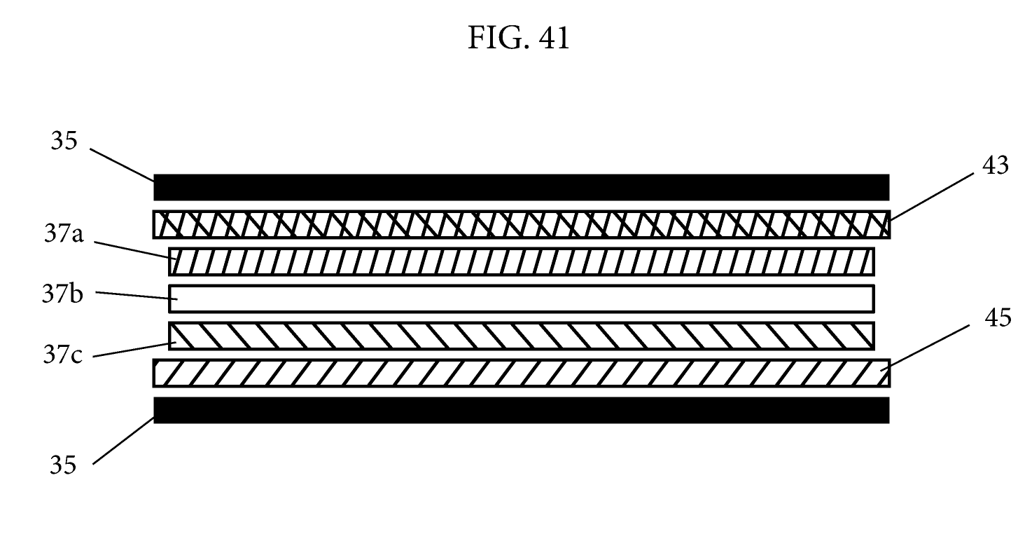

In some embodiments, an impermeable gutter layer 45 may also be included in the insert 10 or band 50, wherein the impermeable gutter layer 45 may be located under the core layers 37 and act to pick up excess sweat that the core layers 37 cannot contain. Therefore, the impermeable gutter layer 45 acts as an impermeable barrier that keeps sweat inside the insert 10 or band 50, and it can preferably be included in the insert 10 and band 50 if there is no bottom, liquid impervious layer 40. For example, as illustrated in FIG. 41, a band 50, such as the band 50 illustrated in FIG. 32, may be comprised of a top, moisture wicking layer 35; a top, elastic layer 43; core layers 37a, 37b, 37c; an impermeable gutter layer 45; and a bottom, moisture wicking layer 35.

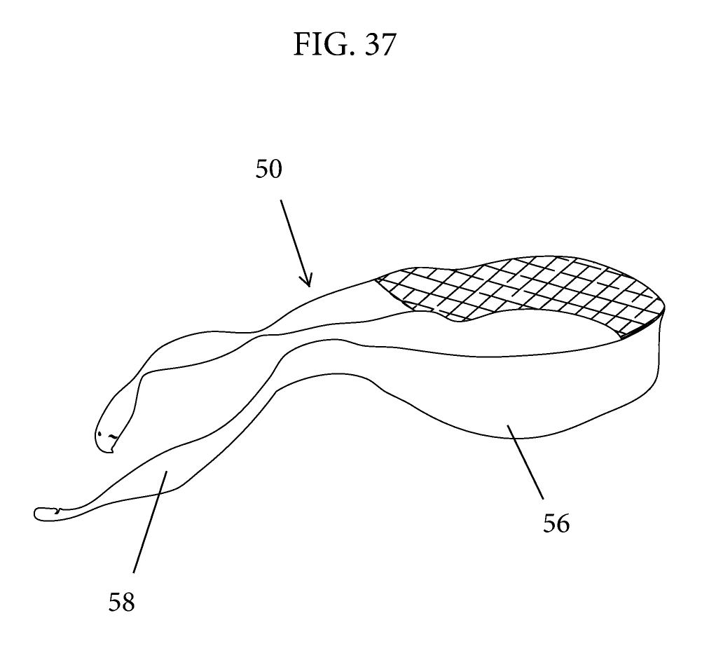

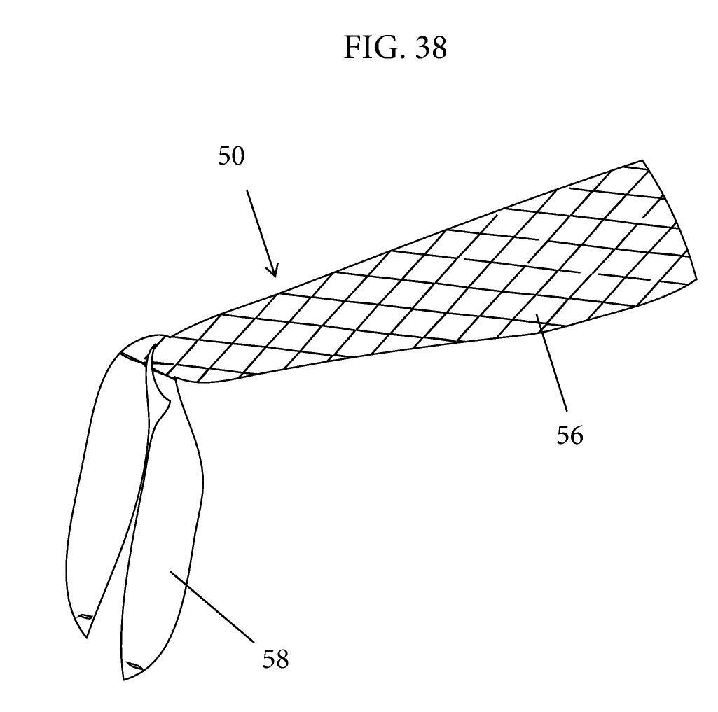

The disposable absorbent insert 10 can be fitted against the front, inside surface of headwear 20, as illustrated in FIGS. 5-12 and 17. The insert 10 can also be fitted against the back, inside surface of headwear 20. In an alternative embodiment, as illustrated in FIGS. 32-38, the disposable insert 10 can be a band 50, such as, but not limited to, a headband, wristband, or sweatband, and it can be positioned around a user's head without the need to be adhered to headwear 20, such as a helmet, hat, or hard hat.

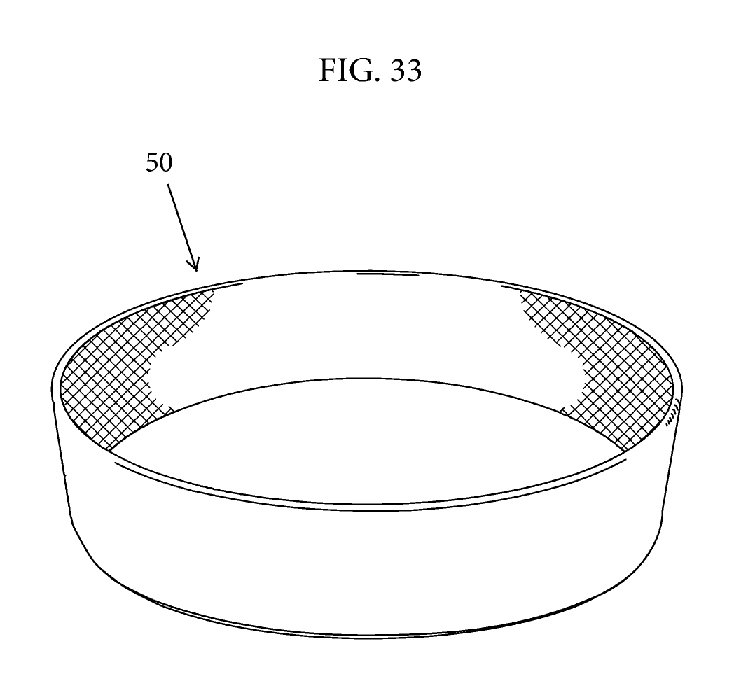

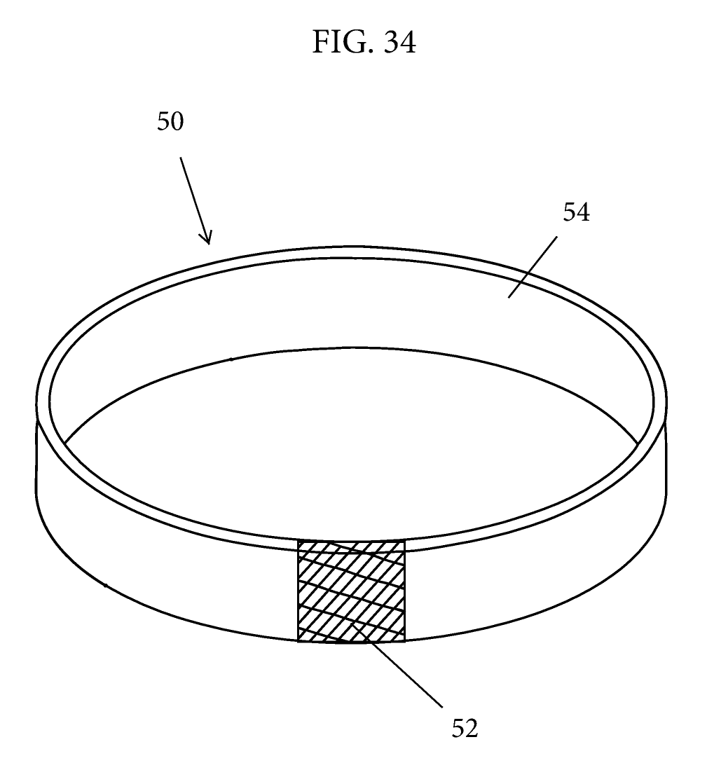

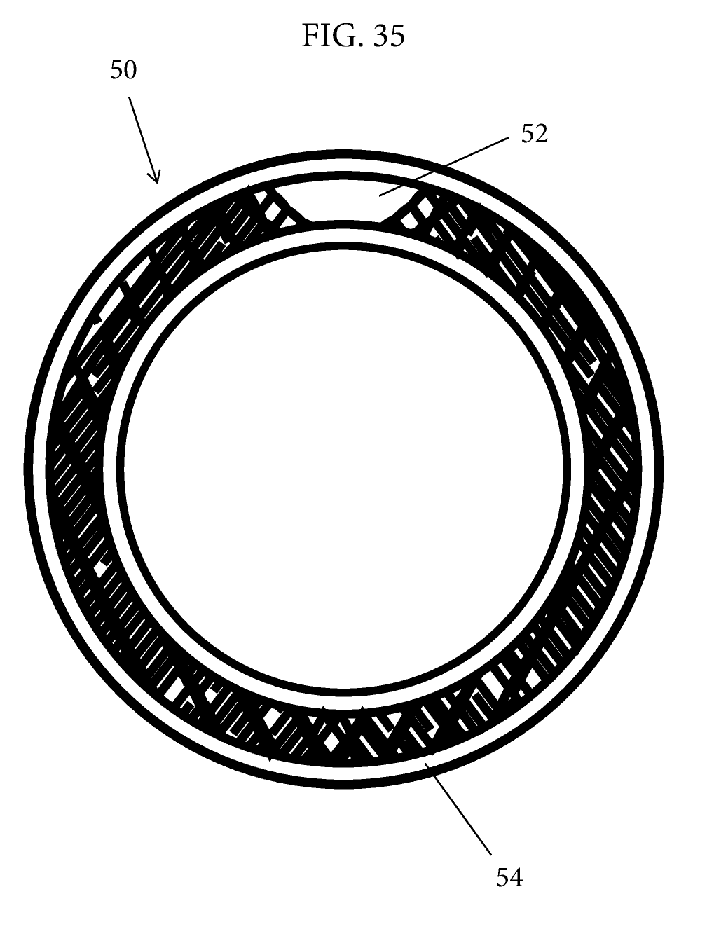

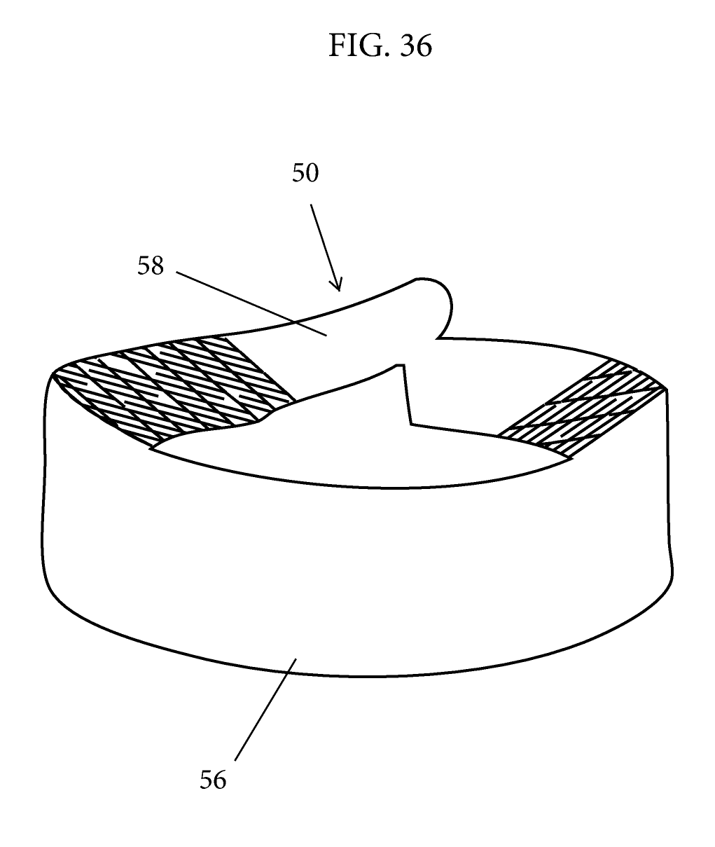

In one embodiment, the device for absorbing and retaining sweat can be an entirely elastic band 50, as illustrated in FIGS. 32 and 33. In another example, the device can be a band 50 that has at least a portion that is elastic 52 as well as an absorbent portion 54, as illustrated in FIGS. 34 and 35. In another embodiment, the device can be a band 50 that is tied onto a user's head, as illustrated in FIGS. 36-38. In this example, the band 50 can be comprised of an absorbent portion 56 and a tie portion 58 so that the user can tie the band 50 as tight as he or she wishes. The band 50 functions to wick moisture away from the head of the user and prevent moisture from dripping down into the eyes, nose, mouth and overall facial area of the user. The band 50 can be in direct contact with the user's head and hair.

In some embodiments, the insert 10 or band 50 can be formed of an absorbent material and can be comprised of several layers, as illustrated in FIGS. 14-16. In one embodiment, the insert 10 or band 50 is comprised of a moisture wicking layer 35 that is in contact with the user's head when in use and wicks moisture away from the user's head. Underneath the moisture wicking layer 35 may be an absorbent core layer 37 that collects and holds the moisture that has been wicked away from the user's head. Underneath the absorbent core layer 37 may be a bottom, liquid impervious layer 40 that, along with the moisture wicking layer 35, encases the absorbent core layer 37 and prohibits moisture from leaving the absorbent core layer 37.

In some embodiments, underneath the bottom, liquid impervious layer 39 is a pressure sensitive adhesive layer 41 that covers the entire span of a bottom-side of the bottom, liquid impervious layer 40. In another embodiment, only a portion of the bottom, liquid impervious layer 40 is covered by the pressure sensitive adhesive layer 41. The pressure sensitive adhesive 41 enables the insert 10 to adhere to the surface on the inside of the headwear 20. The adhesive of layer 41 is a repositionable pressure sensitive adhesive (and may take the form of double-sided tape), which allows insert 10 to be removed and discarded from headwear 20 (or a suspension headband) without leaving adhesive residue. A removable release liner layer 39 covers the adhesive layer 41 prior to use and is removed to expose the adhesive layer 41 for mounting the insert 10 within the headwear 20. In one embodiment, the release liner layer 39 is release paper. In another embodiment, the release liner layer 39 is a material with silicon one or both sides of the release liner layer 39.

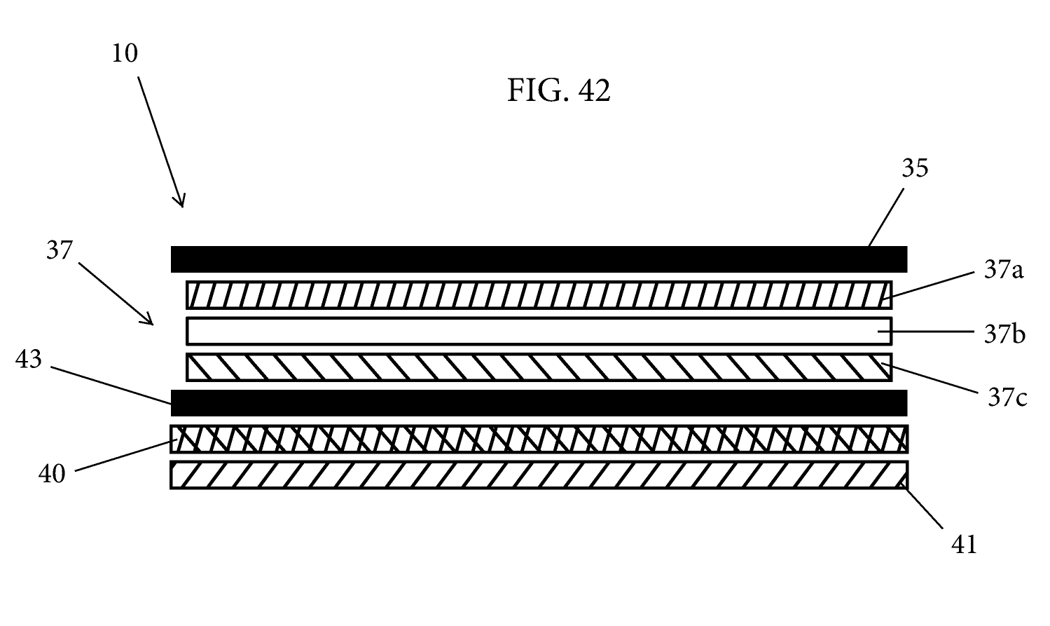

In some embodiments, as seen in FIGS. 13-16 and 39-42, the insert 10 or band 50 is comprised of a moisture wicking layer 35 that is in contact with the user's head when in use and wicks moisture away from the user's head. Below the moisture wicking layer 35 is an absorbent core layer 37 that is comprised of a top absorbent core layer 37a, a middle, super absorbent core layer 37b, and a bottom absorbent core layer 37c. The absorbent core layer 37 collects and holds the moisture that has been wicked away from the user's head. In some embodiments, underneath the absorbent core layer 37 is a liquid impervious layer 40 that, along with the moisture wicking layer 35, encases the absorbent core layer 37 and prohibits moisture from leaving the absorbent core layer 37. In some embodiments, an additional, elastic layer 43 is included that enables the insert 10 to have elasticity. Therefore, in one example, the insert 10 may have a top, moisture wicking layer 35; an absorbent core layer 37 comprised of a top absorbent core layer 37a, a super absorbent core layer 37b, and a bottom absorbent core layer 37c; an elastic layer 43; a bottom, liquid impervious layer 40; and a pressure-sensitive adhesive layer 41, as illustrated in FIG. 42.

As described above, underneath the bottom, liquid impervious layer 40 can be a pressure-sensitive adhesive layer 41 that covers part of, or the entirety of, a bottom-side of the bottom, liquid impervious layer 40, thus enabling the insert 10 to adhere to the surface on the inside of the head covering 20. The adhesive layer 41 may have a removable release liner layer 39 that covers the adhesive layer 41 prior to use and can be removed to expose the adhesive layer 41 for mounting.

In some embodiments, there is no liquid impervious layer 40, as illustrated in FIGS. 39-41. For example, one embodiment of the band 50, illustrated in FIG. 39, is comprised of five layers: a top, moisture wicking layer 35; an absorbent core layer 37 comprised of a top absorbent core layer 37a, a super absorbent core layer 37b, and a bottom absorbent core layer 37c; and a second, moisture wicking layer 35.

In another example, one embodiment of the band 50, illustrated in FIG. 40, is comprised of seven layers: a top, moisture wicking layer 35; a first elastic layer 43; an absorbent core layer 37 comprised of a top absorbent core layer 37a, a super absorbent core layer 37b, and a bottom absorbent core layer 37c; a second elastic layer 43; and a second, moisture wicking layer 35.

In another example, one embodiment of the band 50, illustrated in FIG. 41, is comprised of seven layers: a top, moisture wicking layer 35; an elastic layer 43; an absorbent core layer 37 comprised of a top absorbent core layer 37a, a super absorbent core layer 37b, and a bottom absorbent core layer 37c; a impermeable gutter layer 45; and a second, moisture wicking layer 35.

In some embodiments, the moisture wicking layer 35, the liquid impervious layer 40, or both can incorporate flame-resistant fibers (e.g., Nomex by DuPont). In some embodiments, the device for absorbing and retaining sweat can include an absorption indicator or a saturation indicator, such as a color change indicator, wherein the color changes as more sweat is absorbed, and the change in color enables the user to know how much sweat the device has absorbed and about how much more it can absorb. In some embodiments, the absorption or saturation indicator can be a strip on the device. In one embodiment, the device can include glow in the dark materials so that the device is visible in darkness.

In some embodiments, in order to use the insert 10, the removable release liner layer 39 is removed from the adhesive layer 41 by peeling it from the adhesive layer 41. The insert 10 is then applied to the headwear 20, either by applying it directly to interior surfaces of the headwear shell itself, or to a suspension headband shaped to conform to a user's head. The repositionable pressure sensitive adhesive of the adhesive layer 41 holds the insert 10 in place. The gaps 30 in the insert 10 facilitate bending of the insert 10 to conform to the shape of the headwear 20, as illustrated in FIGS. 5-12, and to prevent overlapping of the material of the insert 10. By preventing such overlap, a smooth surface of insert 10 lies against the user's head for increased comfort. After installation, the insert 10 can be firmly secured to the headwear 20 by the repositionable pressure sensitive adhesive 41, with the exposed surface of the insert 10 forming a contacting surface for the wearer of the headwear 20. After use, the insert 10 is easily removed by breaking the adhesive seal between the insert 10 and the curved inside surface of the headwear 20.

In one embodiment, the insert 10 may be an absorbent cylinder that can be elastic. The cylinder can include three layers: a top layer, an airlaid layer, and a SAP layer. The SAP layer can be in the middle of the cylinder and the layers can be rolled up like a tube. In one embodiment, several cylinders or tubes can be intertwined or braided together to make a larger end product.

In one embodiment, an insert 10 may contain one or more vents or cutouts that are used for ventilation and circulation. The vents or cutouts may be any shape, such as, but not limited to, a linear slit, a rectangle, a non-linear shape, an oval, etc. Further, the edges of the vents or cutouts may each have a sealed perimeter, similar to that described above.

Although the device for absorbing and retaining sweat disclosed herein has been described with respect to several embodiments, workers skilled in the art will recognize that changes may be made in form and detail without departing from the spirit and scope of the disclosure.

What is claimed is:

1. A disposable absorbent insert for attaching to a headband portion of a suspension system in protective headwear, the insert comprising:
a top, moisture wicking layer comprised of a hydrophilic fabric;
a liquid absorbent, retentive core comprised of three layers, wherein the first layer is a first, absorbent airlaid layer, the second layer is a superabsorbent polymer layer, and the third layer is a second, absorbent airlaid layer;
a liquid impervious layer comprising a polyethylene film;
an adhesive layer attached to, and covering, at least a portion of the liquid impervious layer;
a release liner layer attached to the adhesive layer; and
a continuous seal along an entire perimeter of the insert that is a thickness of from 1 mm to 3 mm;
wherein:
the insert is roughly rectangular and has a long top edge, a long bottom edge, and four rounded corners;
the insert is capable of attaching to a first, forehead-contacting side of the headband portion of the suspension system.

2. The disposable absorbent insert of claim 1, further comprising a first tab, wherein the first tab protrudes from the long top edge of the insert and is capable of folding over a first edge of the headband portion of the suspension system and adhering to a second side of the headband portion of the suspension system.

3. The disposable absorbent insert of claim 2, wherein the first tab is a first long tab that is evenly centered along the long top edge.

4. The disposable absorbent insert of claim 3, further comprising a second long tab that protrudes from the long bottom edge of the insert, is evenly centered along the long bottom edge, and is capable of folding over a second edge of the headband portion of the suspension system and adhering to the second side of the headband portion of the suspension system.

5. The disposable absorbent insert of claim 4, wherein the second long tab attaches to the first long tab.

6. The disposable absorbent insert of claim 5, wherein the first and second long tabs are hook and loop tabs.

7. The disposable absorbent insert of claim 5, wherein the first and second long tabs are snap tabs.

8. The disposable absorbent insert of claim 2, further comprising a second tab, wherein the first and the second tabs are short tabs and are evenly centered along the long top edge, and wherein the second tab protrudes from the long top edge of the insert, and is capable of folding over the first edge of the headband portion of the suspension system and adhering to the second side of the headband portion of the suspension system.

9. The disposable absorbent insert of claim 8, further comprising a third short tab and a fourth short tab that protrude from the long bottom edge of the insert, are evenly centered along the long bottom edge, and are capable of folding over a second edge of the headband portion of the suspension system and adhering to the second side of the headband portion of the suspension system.

10. The disposable absorbent insert of claim 9, wherein the third short tab attaches to the first short tab and wherein the fourth short tab attaches to the second short tab.

11. The disposable absorbent insert of claim 10, wherein the first, second, third, and fourth short tabs are hook and look tabs.

12. The disposable insert of claim 10, wherein the first, second, third, and fourth short tabs are snap tabs.

13. The disposable absorbent insert of claim 2, further comprising a second and third tab, wherein the first and second tabs are short tabs, the third tab is a first long tab, the first short tab is located on a first end of the long top edge, the second short tab is located on a second end of the long top edge, and the first long tab is centered along the long top edge.

14. The disposable absorbent insert of claim 13, further comprising a third short tab, a fourth short tab, and a second long tab, wherein the third short tab is located on a first end of the long bottom edge, the fourth short tab is located on a second end of the long bottom edge, and the second long tab is centered along the long bottom edge.

15. The disposable absorbent insert of claim 14, wherein the third short tab attaches to the first short tab, the fourth short tab attaches to the second short tab, and the second long tab attaches to the first long tab.

16. The disposable absorbent insert of claim 15, wherein the first, second, third, and fourth short tabs and the first and second long tabs are hook and loop tabs.

17. The disposable absorbent insert of claim 15, wherein the first, second, third, and fourth short tabs and the first and second long tabs are snap tabs.

18. The disposable absorbent insert of claim 2, wherein the first tab is an adhesive tab.

19. The disposable absorbent insert of claim 2, wherein the first tab is a hook and loop tab.

20. The disposable absorbent insert of claim 2, wherein the first tab is a snap tab.

21. The disposable absorbent insert of claim 1, further comprising an elastic layer located between the liquid absorbent, retentive core and the liquid impervious layer.

22. The disposable absorbent insert of claim 1, wherein an impermeable gutter layer replaces the liquid impervious layer.

23. The disposable insert of claim 1, wherein a moisture wicking layer replaces the liquid impervious layer.

24. The disposable absorbent insert of claim 1, wherein at least one of the top, moisture wicking layer and the liquid impervious layer is further comprised of flame-resistant fibers.

25. The disposable absorbent insert of claim 1, wherein at least one of the top, moisture wicking layer and the liquid impervious layer is further comprised of foam.

26. The disposable absorbent insert of claim 25, wherein the foam is porous.

27. The disposable absorbent insert of claim 1, wherein the liquid absorbent, retentive core is further comprised of a superabsorbent retentive foam material.

28. The disposable absorbent insert of claim 1, further comprising a saturation indicator, wherein the color of the saturation indicator changes as more moisture is absorbed by the disposable absorbent insert.

29. The disposable absorbent insert of claim 1, wherein the adhesive layer attaches to, and covers, the entirety of the liquid impervious layer.

30. The disposable absorbent insert of claim 1, wherein at least one of the top, moisture wicking layer, the liquid absorbent, retentive core, and the liquid impervious layer is further comprised of recycled materials.

31. The disposable absorbent insert of claim 1, wherein the insert is further comprised of biodegradable material.

32. The disposable absorbent insert of claim 1, further comprising one or more vents.

33. The disposable absorbent insert of claim 32, wherein each vent has a sealed perimeter.

34. A disposable absorbent insert for fitting against a forehead-contacting, curved inside surface of a user's athletic head covering, the insert comprising:
a top, moisture wicking layer comprised of a hydrophilic fabric;
a liquid absorbent, retentive core comprised of three layers, wherein the first layer is a first, absorbent core layer, the second layer is a superabsorbent core layer, and the third layer is a second, absorbent core layer;
a liquid impervious layer;
an adhesive layer attached to the liquid impervious layer;
a release liner layer attached to the adhesive layer;
a continuous seal along an entire perimeter of the insert that is a thickness of from 1 mm to 3 mm;
a lower, curved edge;
two rounded end edges;
a top edge comprised of a first outer, top edge portion, a second outer, top edge portion, and an inner, top edge portion, wherein the first and second outer, top edge portions are positioned on either side of the inner, top edge portion;
a first gap located between the first outer, top edge portion and the inner, top edge portion, the first gap extending at a 40 to 50 degree angle from the top edge of the insert toward a central portion of the insert; and
a second gap located between the second outer, top edge portion and the inner, top edge portion, the second gap extending at a 40 to 50 degree angle from the top edge of the insert toward the central portion of the insert.

35. The disposable absorbent insert of claim 34, wherein at least one of the top, moisture wicking layer and the liquid impervious layer is further comprised of flame-resistant fibers.

36. The disposable absorbent insert of claim 34, wherein at least one of the top, moisture wicking layer, the liquid absorbent, retentive core, and the liquid impervious layer, is further comprised of foam.

37. The disposable absorbent insert of claim 34, wherein the adhesive layer covers an entire surface of the liquid impervious layer.

38. The disposable absorbent insert of claim 34, further comprising a saturation indicator, wherein the color of the saturation indicator changes as more moisture is absorbed by the disposable absorbent insert.

39. The disposable absorbent insert of claim 34, wherein at least one of the top, moisture wicking layer, the liquid absorbent, retentive core, and the liquid impervious layer is further comprised of recycled materials.

40. The disposable absorbent insert of claim 34, wherein the insert is further comprised of biodegradable material.

41. The disposable absorbent insert of claim 34, further comprising one or more vents.

42. The disposable absorbent insert of claim 41, wherein each vent has a sealed perimeter.
